(19) United States
(12) Patent Application Publication
(10) Pub. No.: US 2005/0266438 A1
(43) Pub. Date: Dec. 1, 2005

(54) Genetic networks regulated by attenuated GH/IGF1 signaling and caloric restriction

(75) Inventors: Stephen R. Spindler, Riverside, CA (US); Tomoshi Tsuchiya, Oita-city (JP); Joseph M. Dhahbi, Alameda, CA (US); Andrzej Bartke, Mechanicsburg, IL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California, Oakland, CA (US); The Board of Trustees of Southern Illinois University, Springfield, IL (US)

(21) Appl. No.: 11/083,538

(22) Filed: Mar. 16, 2005

Related U.S. Application Data
(60) Provisional application No. 60/553,689, filed on Mar. 16, 2004.

(51) Int. Cl. C12Q 1/68; A61K 49/00

(52) U.S. Cl. 435/6; 424/9.2; 800/3

(57) ABSTRACT

The invention is based on the discovery that the growth hormone-insulin-like growth factor-1 genetic signaling pathway and caloric restriction in conjunction extend lifespan and delay the onset of age-related diseases.

### CROSS-REFERENCE TO RELATED APPLICATIONS

This application claims benefit of U.S. Application No. 60/553,689, filed Mar. 16, 2004, which application is herein incorporated by reference.

### STATEMENT AS TO RIGHTS TO INVENTIONS MADE UNDER FEDERALLY SPONSORED RESEARCH OR DEVELOPMENT

This invention was made, in part, with Government support under Grant No. AG19899, awarded by the National Institutes of Health. The government has certain rights in this invention

### BACKGROUND OF THE INVENTION

The invention is based on the discovery that the growth hormone-insulin-like growth factor-1 genetic signaling pathway and caloric restriction in conjunction extend lifespan and delay the onset of age-related diseases.

Genetic ablation of growth hormone (GH) or its receptor, and suppression of plasma concentration of insulin-like growth factor-1 (IGF1) produce a dwarf phenotype (DF) and extend the lifespan of rodents (Longo & Finch Science 299:1342-1346, 2003). Ames DF mice, which are homozygous for a loss of function mutation at the Prop 1 locus, exhibit a 40 to 70% increase in mean and maximal lifespan compared with their normal heterozygous siblings (Brown-Borg, et al., Nature 384:33, 1996). Several lineages of anterior pituitary cells do not develop normally in these mice, leading to a combination of endocrine abnormalities, including low levels of GH/IGF 1, thyroid-stimulating hormone, thyroid hormones, and prolactin (Sornson, et al., Nature 384:327-333, 1996). DF postpones the age-related development of neoplastic diseases, immune system decline, and collagen cross-linking, suggesting DF reduces the rate of aging (Ikeno, et al., J. Gerontol. A Biol. Sci. Med. Sci. 58:291-296, 2003; Flurkey, et al., Proc. Natl. Acad. Sci. U.S. A 98:6736-6741, 2001). Decreased IGF1 signaling is thought to exert the major influence on longevity. GH receptor knockout mice have significantly extended lifespan, but IGF1 receptor knock out mice also have extended lifespan, 90% reduced levels of IGF1, and high levels of plasma GH (Coschigano, et al., Endocrinology 141:2608-2613, 2000; Holzenberger, et al., Nature 421:182-187, 2003; Zhou, et al., Proc. Natl. Acad. Sci. U.S. A 94:13215-13220, 1997).

Caloric restriction (CR), which is reduced caloric consumption without malnutrition, retards aging and most disease processes, and increases maximum and/or mean lifespan in a variety of organisms (Koubova & Guarente, Genes Dev. 17:313-321, 2003). CR and DF together additively increase the lifespan of mice (Bartke, et al., Nature 414:412, 2001). These effects could be mediated through one pathway which is more strongly affected by the combined interventions, or through distinct molecular pathways independently affected by each intervention. The shape of the lifespan curves suggested to Bartke et al. that different molecular mechanisms were responsible for the additive lifespan effects of DF and CR (Bartke, et al., Nature 414:412, 2001). In contrast, Clancy and colleagues suggested that overlapping mechanisms mediate these effects (Clancy, et al., Science 296:319, 2002). In Drosophila, mutation of the chico gene, which encodes a homolog of mammalian insulin receptor substrates 1 through 4, reduces insulin/IGF1 signaling and results in a DF phenotype with extended lifespan. These authors speculated that CR and chico utilize overlapping mechanisms. Shimokawa and colleagues observed an additive lifespan effect of DF and CR in mini-rats overexpressing antisense GH RNA (Shimokawa, et al., FASEB J 17:1108-1109, 2003). They concluded that CR affects aging and longevity mostly through mechanisms other than suppression of the GH-IGF1 axis. None of the studies provided strong evidence indicating whether life-prolonging effects of DF and CR are mediated by distinct or overlapping molecular mechanisms.

A major goal of pharmaceutical research has been to discover ways to reduce morbidity and delay mortality. CR remains the most reliable intervention capable of consistently extending lifespan and reducing the incidence and severity of many age-related diseases, including cancer, diabetes, and cardiovascular disease. Dwarfism appears to be a second such intervention. Additionally, physiological biomarkers linked to lifespan extension in rodents (e.g., mice, rats), other mammals (e.g., rabbits) and monkeys that have been subjected to CR have been shown to be associated with extended lifespan in humans; see for examples, Weyer, et al., Energy Metabolism after Two Years of Energy Restriction: the Biosphere Two Experiment, Am. J. Clin. Nutr. 72, 946-953, 2000, and Roth, et al., Biomarkers of Caloric Restriction may Predict Longevity in Humans, Science 297, 811, 2002. These preliminary findings suggest that the anti-aging effects of CR and dwarfism may be universal among all species.

Thus, there is a need to identify genetic pathways that mediate anti-aging effects, e.g., those induced by caloric restriction and dwarfism, and to determine whether such genetic pathways are overlapping or distinct. This invention addresses that need.

### BRIEF SUMMARY OF THE INVENTION

In one aspect, this invention is based on the discovery that dwarfism and caloric restriction in conjunction extend lifespan and delay the onset of age-related diseases.

The invention therefore provides a method of identifying an intervention that modulates a biomarker of aging, the method comprising: exposing a biological sample to a test intervention; measuring the level of at least one gene product set forth in Table 3; and identifying a change in the level of the gene product that correlates with a change observed in dwarfism, caloric-restriction or both caloric-restriction and dwarfism, thereby identifying an intervention that modulates a biomarker of aging. In some embodiments, the biological sample is an animal, e.g., a mouse. The biological sample that is treated with the test intervention can also be cells, e.g., liver cells, isolated from a mammal.

The method can be practiced by evaluating the level of one or more gene products. Often, the expression pattern is evaluated for multiple gene products. The one or more gene products can be generally involved in the same biological pathway, or different pathways. For example, a gene product can be a member of a signal transduction cascade, or play a role in apoptosis, glucose metabolism, lipid metabolism, or oxidant and toxin defense. In one embodiment, the gene product is a chaperone.

In some embodiments, the step of measuring the level of the gene product comprises measuring the level of mRNA, for example by using an oligonucleotide array or another method such as polymerase chain reaction (PCR). Optionally, the method can comprise an additional step of determining mRNA level using an alternative techniques. In an exemplary embodiment, mRNA is determined using an oligonucleotide array and an additional method, e.g., PCR.

In other embodiments, the step comprises measuring the level of protein, measuring protein activity, or measuring protein modification in response to the test intervention. Protein modifications includes phosphorylation, sulfation, glycosylation, ubiquitination, or any other modification of proteins.

In another aspect, the invention provides a method of identifying a biomarker of aging, the method comprising: comparing an expression profile from a caloric-restricted dwarf mouse to the expression profile from a control-fed normal mouse and a control-fed dwarf mouse, and identifying changes in the expression profile that occur in the caloric-restricted dwarf mouse relative to the control-fed normal and dwarf mice. In some embodiments, the method further comprises comparing the expression profile in the caloric-restricted dwarf mouse to an expression profile from a normal mouse subjected to caloric restriction and identifying those changes that occur in the caloric-restricted dwarf mouse relative to the caloric-restricted normal mouse.

In one embodiment the step of comparing the expression profile from the caloric restricted mouse to that of the control-fed mice comprises measuring levels of RNA, e.g., using an oligonucleotide-based high density array. In other embodiments, the step of determining the expression profile from the caloric-restricted mouse to that of the expression profile of the control-fed mice comprises measuring levels of protein, protein activity, or protein modification. The expression profile can be obtained by evaluating any tissue, for example, liver tissue or heart tissue.

In one embodiment, the dwarf mouse is subjected to short-term caloric restriction, e.g., for about eight weeks or less; often, four weeks or less; or for short times such as about 2 days or 1 day.

In another aspect, the invention provides a method of identifying an intervention that modulates a biomarker of longevity, the method comprising: exposing a biological sample to a test intervention; measuring the level of a gene product identified in accordance with the method set forth above and identifying a change in the level of the gene product that mimics that observed in a dwarf mouse, a caloric-restricted mouse, or a dwarf mouse that is caloric-restricted relative to a control-fed normal or dwarf mouse, thereby identifying an intervention that modulates a biomarker of longevity. In some embodiments, the change in the level of the gene product is determined using an oligonucleotide-based high density array.

The invention also provides a method of identifying a biomarker of aging, the method comprising comparing an expression profile of a biological sample from a dwarf mouse to the gene expression profile from a control-fed normal mouse and a caloric-restricted normal mouse, and identifying changes in the expression profile that occur in the dwarf mouse relative to the control-fed and caloric-restricted normal mice. In some embodiments, the method further comprises comparing the expression profile from the dwarf mouse to that of a caloric-restricted dwarf mouse.

The invention also provides a method of identifying a biomarker of aging, the method comprising: comparing an expression profile of a biological sample from a caloric-restricted normal mouse to the gene expression profile from a control-fed dwarf mouse and a control-fed normal, and identifying changes in the expression profile that occur in the caloric-restricted mouse relative to the control-fed dwarf and normal mice.

### BRIEF DESCRIPTION OF THE DRAWINGS

FIGS. 1A and 1B provides a numerical summary of hepatic gene expression profiling of normal and DF mice fed AL or CR as described in the Examples section. Panel A, DF changed the expression of 313 genes (213+100 genes), CR changed the expression of 177 genes (77+100), and DF and CR together changed the expression of 390 genes (213+100+77 genes), 100 of which were additively changed in expression. Of the additively changed genes, 95 showed no statistical evidence of interaction between DF and CR, while 5 showed evidence of interaction. Panel B, a model for the regulation of 213 genes by DF (hypothetical gene 1), 77 genes by CR (hypothetical gene 2), 95 genes independently and additively by CR and DF (hypothetical gene 3), and 5 genes for which diet and genotype interact to regulate expression (hypothetical gene 4).

FIG. 2 shows an expression analysis of 16 genes measured using Affymetrix microarrays and qPCR. Solid and open bars represent microarray or qPCR data, respectively. The fold change for microarray and qPCR were calculated as described herein in the Examples section. Genes are identified by their GenBank numbers.

### DETAILED DESCRIPTION OF THE INVENTION

The invention provides methods of identifying biomarkers of caloric restriction and methods of identifying mimetics of caloric restriction. Such mimetics can be used, e.g., for extending lifespan or delaying or mitigating the effects of age-related diseases, e.g., cancer, cardiovascular disease and the like.

Definitions

The term “expression pattern” as used herein refers to the level of a product encoded by one or more gene(s) of interest. A product can be a nucleic acid or protein. The “expression level” as used herein refers to the amount of the product as well as the level of activity of the product. Accordingly, the expression level can be determined by measuring any number of endpoints. These endpoints include amount of mRNA, amount of protein, amount of protein activity, protein modifications, and the like. Often, e.g., when the expression pattern of multiple gene products is evaluated, the term is used interchangeably with “expression profile”.

A “dwarf mouse” refers to a mouse that has a deficiency in the growth hormone/growth hormone receptor and/or insulin-like growth factor-1 (IGF-1) recpetor pathway. Such mice include well known genetic models such as the Ames dwarf mouse, the Snell mouse, a mouse having the little mutation, etc. Typically, several lineages of anterior pituitary cells do not develop normally in dwarf mice, leading to a combination of endocrine abnormalities, including low levels of GH/IGF1, thyroid-stimulating hormone, thyroid hormones, and prolactin.

“Caloric restriction” as used herein refers to a diet in which the amount of calories is reduced in comparison to a normal diet without malnutrition. Typically, a caloric restricted diet constitutes about 90% or 85%, often 80%, 75%, 70%, 65%, 60%, 55%, or 50% of a normal diet for a subject. As appreciated by one of skill in the art, a normal diet is determined with respect to factors such as age, sex, height and body frame, and the like. Examples of normal diets in animals are known in the art, e.g, set forth by the Subcommittee on Laboratory Animal nutrition and Committee on Animal Nutrition in Nutrient Requirements of Laboratory Animals: rat, mouse, gerbil, guinea pig, hamster, vole, fish (Natl. Acad. Sci, Washington, D.C. pp 38-50, 1978)

“Short-term caloric restriction” refers to a period of caloric restriction that is less than the adult life of an animal. Typically, short-term caloric restriction as used in the methods described herein ranges from about 1 day to about 2 months. Exemplary periods of short-term caloric restriction include about 1 day, 2 days, 1 week, 2 weeks, three weeks, four weeks, 6 weeks, 8 weeks, or 12 weeks.

The term “caloric-restricted sequence” or “biomarker of caloric restriction” refers to a nucleic acid and/or protein sequence that is differentially expressed in caloric-restricted, and/or dwarf mice. A “biomarker of longevity” as used herein refers to a nucleic acid and/or protein sequence that is associated with longevity. In the current invention, such a biomarker is differentially expressed in caloric-restriction and/or dwarfism relative to normal caloric restriction and normal growth hormone/IGF signal transduction. Caloric-restricted sequences include those that are up-regulated (i.e., expressed at a higher level) in caloric-restriction, as well as those that are down-regulated (i.e., expressed at a lower level).

“Up-regulation” as used herein means that the ratio of the level of product in treated vs. control is greater than one. Often, the ratio is 1.1, 1.3, 1.5, 2.0 or greater. As appreciated by those in the art, statistical analysis is typically performed to evaluate significance.

“Down-regulation” as used herein means that the ratio of the level of product in treated vs. control is less than one. Often the ratio is 0.75, 0.5, 0.25 or less. As appreciated by those in the art, statistical analysis is typically performed to evaluate significance.

“Treated” refers to a biological sample that is subjected to caloric-restriction or a candidate intervention that mimics caloric restriction and/or longevity associated with dwarfism.

The term “biological sample” encompasses a whole organism as well as cells or tissues isolated from the organism. The biological sample is often mammalian, typically a rodent, such as a mouse, rat, hamster, guinea pig etc. However, other mammalian biological samples can be used, including humans and non-human primates such as monkeys.

A “CR mimetic” refers to a compound, a test compound, an agent, a pharmaceutical agent, or the like, that reproduces at least some effects induced by CR, in normal or dwarf animals. It is to be appreciated by one skilled in the art that the exemplary methods are not limited to analyzing changes in RNA levels that are affected by CR or CR mimetics but may include changes in physiological biomarkers such as changes in protein levels, protein activity, nucleic acid levels, carbohydrate levels, lipid levels, the rate of protein or nucleic acid synthesis, protein or nucleic acid stability, protein or nucleic acid accumulation levels, protein or nucleic acid degradation rate, protein or nucleic acid structures or functions, and the like.

An “intervention” refers to a treatment regimen or protocol that stimulates an expression pattern that mimics that associated with caloric restriction, dwarfism, or both states.

Introduction

This invention is based, in part, on the discovery that the growth hormone-insulin-like growth factor-1 genetic signaling pathway and caloric restriction in conjunction extend lifespan and delay the onset of age-related diseases. The invention also is based on the discovery that both independent and dependent genetic pathways mediate longevity in caloric-restriction and dwarfism. The invention therefore provides methods of identifying biomarkers of longevity that are associated with caloric-restriction, dwarfism, and both caloric-restriction and dwarfism. Such biomarkers can be used to identify interventions that mimic the expression pattern of one or more of such biomarkers in caloric-restriction and/or dwarfism. Such interventions can be used, e.g., to extend lifespan.

The practice of the present invention often employs conventional molecular biology and recombinant DNA techniques, which are commonly known in the art. Such techniques are described, e.g., in Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-1999. Other sources of techniques for evaluating expression patterns, e.g., protein level, protein activity, or protein modification are also known in the art. Such techniques can also be found in relevant sections (i.e., those that may relate to the particular protein or modification being evaluated) of references such as the Methods in Enzymology series (Academic Press).

The screening assays to identity biomarkers of longevity described herein are typically performed using mammalian systems, e.g., cells isolated from a mammalian subject and/or mammals. However, in some embodiments, such as screening candidate interventions, non-mammal organisms such as insects, nematodes, yeast, bacteria, and other organisms may also be used. In some embodiments, evaluation of candidate drugs can be performed in these non-mammal organisms and then subsequently tested in mammals (e.g., mice or humans).

Expression Patterns

In certain embodiments, longevity-associated sequences are identified using expression patterns. An expression pattern of a particular sample is essentially a “fingerprint” of the state of the sample. Typically, an expression pattern is obtained by measuring the products of two or more genes. The evaluation of a number of gene products simultaneously allows the generation of an expression patterns that is characteristic of caloric restriction, dwarfism, or both caloric-restriction and dwarfism. By comparing expression profiles of caloric-restricted and/or dwarf animals to control-fed animals and normal animals, information regarding which genes are important (including both up- and down-regulation of genes) in caloric-restriction and longevity is obtained.

Expression profiles can be generated for that population of product using any tissue or organ that is subjected to caloric-restriction or a test intervention. In one embodiment, expression profiles are generated for genes expressed in the liver.

“Differential expression,” or grammatical equivalents as used herein, refers to qualitative or quantitative differences in the temporal and/or cellular expression patterns within and among cells and tissue. Thus, a differentially expressed gene can qualitatively have its expression (e.g., nucleic acid and/or protein expression levels) and/or activity altered, including an activation or inactivation, in, e.g., tissue from normal-fed versus caloric-restricted animals. Some genes will be expressed in one state or cell type, but not in both. Alternatively, the difference in expression may be quantitative, e.g., in that expression is increased or decreased; i.e., gene expression is either upregulated, resulting in an increased amount of transcript or protein or protein activity, or downregulated, resulting in a decreased amount of transcript or protein or protein activity. The degree to which expression differs need only be large enough to quantify via standard characterization techniques. For example, nucleic acid levels can be determined using Affymetrix GeneChip™ expression arrays (e.g., Lockhart, Nature Biotechnology 14:1675-1680, 1996), as outlined below, e.g., for the evaluation of nucleic acid levels in dwarfism and/or caloric restriction. Other techniques for analyzing levels of nucleic acids include, but are not limited to, quantitative reverse transcriptase PCR, northern analysis and RNase protection.

The effects of CR, dwarfism, CR mimetics, and other candidate interventions can be assessed using a variety of assays. Such assays include at least one of the changes in RNA levels, changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structures or function. The effects also include extending the longevity or life span of mammals (e.g., extending the longevity of mice).

Assays for performing such analyses are well known in the art. For example, assay for the activity of a protein activity, e.g., a transcription factor, a kinase, an enzyme involved in glucose metabolism can be performed using a known assay, such as measuring the ability to modulate transcription, modulate phosphorylation, or perform an enzymatic reaction.

Control data can be obtained from a prior study, the results of which are recorded, as opposed to obtaining the control data concurrently, e.g., at the same time a test intervention is being evaluated. For example, control data may be obtained in a previous study by administering a control diet to a normal or dwarf subject. This control data can then be stored for recall in later screening studies for comparison against the results in the later screening studies. The control data can include changes in RNA level in caloric-restricted subjects and/or dwarf subjects, or other types of measurements to evaluate the expression pattern of a gene product, such as determination of protein levels, protein activity, or protein modifications.

Identification Via Homology or Linkage

Additional longevity-associated sequences can be identified by substantial nucleic acid and/or amino acid sequence homology or linkage to the longevity-associated sequences outlined herein. Such homology can be based upon the overall nucleic acid or amino acid sequence, and is generally determined as outlined below, using either homology programs or hybridization conditions.

The longevity-associated nucleic acid and protein sequences of the invention, e.g., the sequences in Table 2, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the longevity-associated genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/unigene/).

Database of Biomarker of Longevity

The longevity-associated gene products of this invention can collectively provide high-resolution, high-sensitivity datasets which can be used in the areas therapeutics and drug development, and other related areas.

Thus, the present invention provides a database that includes at least one set of assay data. The data contained in the database is acquired, e.g., using array analysis, either singly or in a library format. The database can be in substantially any form in which data can be maintained and transmitted, but is typically an electronic database. The electronic database of the invention can be maintained on any electronic device allowing for the storage of and access to the database, such as a personal computer, but is typically distributed on a wide area network, such as the World Wide Web.

A variety of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The invention also provides for the storage and retrieval of a collection of biomarker data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 biomarker data records cross-tabulated with source.

When the biomarker is a peptide or nucleic acid, the invention typically provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

The invention also provides the use of a computer system, such as that described above, which comprises: (1) a computer; (2) a stored bit pattern encoding a collection of gene expression records obtained by the methods of the invention, which may be stored in the computer; and (3) a comparison target, such as a query target.

Screening Assay for Expression Pattern—High Throughput Screening

In some embodiments, the expression pattern of multiple longevity-associated genes in caloric-restricted dwarf and normal animals, or in biological samples exposed to a potential intervention, are assayed using high-throughput technology.

Often, the expression pattern is obtained by monitoring levels of RNA expression, e.g., levels of mRNA. RNA expression monitoring can be performed on a single polynucleotide or simultaneously for a number of polynucleotides. For example, an oligonucletide array may be used. Other methods, e.g., PCR techniques for measurement of gene expression levels can also be used. Often, once a candidate drug or intervention is identified using high throughput analysis, the results is further confirmed using an alternative method of analyzing expression pattern changes. For example, if an oligonucleotide array is used to initially screen a test intervention, those that identify a test compound or intervention that induces an expression pattern that mimics that observed in caloric restriction, dwarfism, or both, another assay such as a PCR assay can be performed to confirm the results.

Nucleic Acid Probes

In one embodiment, nucleic acid probes to biomarker nucleic acid are made. The nucleic acid probes are designed to be substantially complementary to the biomarker nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, from about 10 to about 80 bases, or from about 30 to about 50 bases. That is, generally complements of ORFs or whole genes are not used. In some embodiments, nucleic acids of lengths up to hundreds of bases can be used.

In some embodiments, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

Attachment of the Target Nucleic Acids to the Solid Support

In some embodiments, as noted above, arrays are used in the screening assays. The arrays can, e.g., be generated to comprise probes for multiple biomarkers associated with longevity.

In general, the probes are attached to a biochip in a wide variety of ways, as will be appreciated by those in the art. As described herein, the nucleic acids can either be synthesized first, with subsequent attachment to the biochip, or can be directly synthesized on the biochip.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

Making Arrays

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. One such substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In one embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups. Using these functional groups, the probes can be attached using functional groups on the probes. For example, nucleic acids containing amino groups can be attached to surfaces comprising amino groups, e.g., using linkers as are known in the art; e.g., homo- or hetero-bifunctional linkers as are well known (see, 1994 Pierce Chemical Company catalog, technical section on cross-linkers, pages 155-200). In addition, in some cases, additional linkers, such as alkyl groups (including substituted and heteroalkyl groups) may be used.

Hybridization and Sandwich Assays

Nucleic acid assays can be detected hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, attached to a solid support, and then the labeled probe is added under conditions that allow the formation of a hybridization complex.

A variety of hybridization conditions may be used in the present invention, including high, moderate and low stringency conditions as outlined above. The assays are generally run under stringency conditions which allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with certain embodiments outlined below. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

Detection of Labeled Target Nucleic Acid Bound to Immobilized Probe

One of skill will readily appreciate that methods similar to those in the preceding section can be used in embodiments where the a nucleic acid to be examined is attached to a solid support and labeled probe is used to detect the biomarker nucleic acid.

Amplification-Based Assays

Amplification-based assays can also be used measure the expression level of biomarker sequences. These assays are typically performed in conjunction with reverse transcription. In such assays, a biomarker nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of biomarker RNA. Methods of quantitative amplification are well known to those of skill in the art. Detailed protocols for quantitative PCR are provided, e.g., in Innis et al., PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

Methods of Assaying Protein Expression Levels

The expression levels of multiple proteins can also be performed. Similarly, these assays may also be performed on an individual basis.

Antibodies to the biomarker protein can generated and used in a variety of immunological detection methods well known in the art. (e.g., Methods in Cell Biology: Antibodies in Cell Biology, volume 37 (Asai, ed. 1993); Harlow & Lane, Antibodies: A Laboratory Manual (1988) and Harlow & Lane, Using Antibodies (1999)). For a review of immunological and immunoassay procedures, see Basic and Clinical Immunology (Stites & Terr eds., 7th ed. 1991). Moreover, such immunoassays can be performed in any of several configurations, which are reviewed extensively in Enzyme Immunoassay (Maggio, ed., 1980); and Harlow & Lane, supra.

Methods of Modulating Gene Expression Levels for Therapeutic Purposes

In one aspect, the invention provides methods of extending longevity by modulating the level of biomarkers identified in accordance with the invention. The specific therapeutic effect will depend on the nature of the biomarker sequence (i.e., which organs the sequences are expressed in, the predicted or known function of the polypeptide encoded by the sequence). In some embodiments, biomarker sequence modulated to treat longevity are those set forth described in Table 1.

Methods of Modulating the Activity of Biomarker Proteins for Therapeutic Purposes

In other aspects, this invention provides methods of modulating the activity of biomarkers of this invention. The specific therapeutic effect will depend on the nature of the sequence (i.e., which organs the sequences are expressed in, the predicted or known function of the polypeptide encoded by the sequence).

It will be appreciated by those of skill in the art that the modulation will either comprise reducing or increasing the activity level of a biomarker protein, depending on the change in expression levels that is associated with longevity in caloric-restriction, dwarfism, or both caloric-restriction and dwarfism. For example, when the biomarker is down-regulated in longevity, such state may be reversed by increasing the activity of the gene product in the cell. This can be accomplished using, e.g., a small molecule activator. Alternatively, e.g., when the sequence is up-regulated in longevity, the activity of the endogenous protein is decreased, e.g., by the administration of an inhibitor.

Small molecule inhibitors and activators can be identified using methods described in the following section.

Methods of Screening Candidate Interventions

The present invention provides novel methods of screening for interventions to enhance lifespan.

In other embodiments, having identified genes that undergo changes in expression pattern in caloric restriction or dwarfism, test compounds can be screened for the ability to modulate gene expression. Although this can be done on an individual gene level, typically, the screening analysis is performed by evaluating the effect of drug candidates on a “gene expression profile”. In considering modulation of a single gene, the preferred amount of modulation of the expression level will depend on the original change of the gene expression in normal versus tissue from the caloric-restricted and/or dwarf organism, with changes of at least 10%, 50%, 100-300%, and in some embodiments 300-1000% relative to control. For example, if a gene exhibits a 4-fold increase in response to caloric-restriction, dwarfism, and/or caloric-restriction and dwarfism compared to normal tissue, an increase of about four-fold is often desired; similarly, a 10-fold decrease in response to caloric-restriction, dwarfism, or a combination of the two compared to normal tissue, often a target value of a 10-fold decrease in expression is desirable to be induced by the test compound.

Typically, a test compound is administered to an organism or cells isolated from the organism. By “administration” or “contacting” herein is meant that the candidate agent is administered in such a manner as to allow the agent to act upon the animal or cells.

The term “test compound” or “drug candidate” or “modulator” or grammatical equivalents as used herein describes any molecule, e.g., small organic molecule, protein polysaccharide, polynucleotide, etc., to be tested for the capacity to modulate the expression pattern of one or more biomarkers. Generally, a plurality of different agent concentrations are tested to obtain a differential response to the various concentrations. Typically, one of these concentrations serves as a negative control, i.e., at zero concentration or below the level of detection.

Methods for Determining Whether a Test Compound Modulates Biomarkers of Longevity

As described above, interventions, e.g., test compounds, that modulate the expression of biomarkers of longevity can be identified by testing compounds for an ability to induce expression patterns that reflect those present in caloric-restriction, dwarfism, or both states. Often, such assays are performed by evaluating levels of expression of RNA or protein.

Based on knowledge of the function of the proteins over/underexpressed, one of skill can use methods known to those of skill in the art to measure the activity of such proteins.

Methods for Monitoring Gene/Protein Expression Levels

Screening for the ability of compounds to modulate expression patterns, e.g., individual gene expression levels or individual protein expression levels, can be conducted via any method known to those of skill in the art, including those described herein.

The amount of gene expression may be monitored using nucleic acid probes, or, alternatively, the gene product itself can be monitored, e.g., through the use of antibodies to the protein and standard immunoassays. Proteomics and separation techniques may also allow quantification of expression. In one embodiment, gene or protein expression monitoring of a number of entities, i.e., an expression profile, is monitored simultaneously. Such profiles will typically involve a plurality of those entities described herein.

In one embodiment, probes to biomarkers of longevity are attached to biochips as outlined above for the detection and quantification of biomarkers and expression monitoring is performed. Alternatively, other assays, such as PCR, may be used.

High-Throughput Screening for Gene Transcription, Polypeptide Expression, & Polypeptide Activity

The assays to identify modulators are amenable to high throughput screening. Typical assays detect modulation of gene expression polypeptide expression, and polypeptide activity when test compounds are contacted with a cell isolated from an animal.

High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

Compounds to be Screened in Methods of this Invention

Combinatorial Libraries

In certain embodiments, combinatorial libraries of potential modulators will be screened for an ability to modulate biomarker activity or expression. Conventionally, new chemical entities with useful properties are generated by identifying a chemical compound (called a “lead compound”) with some desirable property or activity, e.g., modulating expression patterns of biomarkers, creating variants of the lead compound, and evaluating the property and activity of those variant compounds.

In some embodiments, the drug screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Proteins and Nucleic Acids as Potential Modulators

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. These can be libraries of bacterial, fungal, viral, and mammalian proteins, e.g., human protein. Particularly useful test compound will be directed to the class of proteins to which the target belongs, e.g., substrates for enzymes or ligands and receptors.

In one embodiment, modulators are peptides of from about 5 to about 30 amino acids, with from about 5 to about 20 amino acids, or from about 7 to about 15. The peptides may be digests of naturally occurring proteins as is outlined above, random peptides, or “biased” random peptides. By “randomized” or grammatical equivalents herein is meant that the nucleic acid or peptide consists of essentially random sequences of nucleotides and amino acids, respectively. Since these random peptides (or nucleic acids, discussed below) are often chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In another embodiment, the library is biased. That is, some positions within the sequence are either held constant, or are selected from a limited number of possibilities. In one embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc.

The compounds tested as modulators can be any small chemical compound, or a biological entity, such as a protein, sugar, nucleic acid or lipid. Alternatively, modulators can be genetically altered versions of the genes. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Pharmaceutical Administration & Compositions

In certain embodiments, the invention provides pharmaceutical compositions comprising the modulators identified through the assays described in the preceding section, combined with a physiologically acceptable excipient.

In one embodiment, a therapeutically effective dose of a modulator oflongevity-associated genes is administered. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals. Thus the methods are applicable to both human therapy and veterinary applications. In a typical embodiment the patient is a mammal, usually a primate, and most typically, the patient is human.

The administration of the modulators of gene products identified in accordance with the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the modulators may be directly applied as a solution or spray.

The pharmaceutical compositions of the present invention comprise a modulator in a form suitable for administration to a patient. In some embodiments, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that modulators (e.g., antibodies, antisense constructs, ribozymes, small organic molecules, etc.) when administered orally, should be protected from digestion. It is also recognized that, after delivery to other sites in the body (e.g., circulatory system, lymphatic system, or the tumor site) the longevity modulators of the invention may need to be protected from excretion, hydrolysis, proteolytic digestion or modification, or detoxification by the liver. In all these cases, protection is typically accomplished either by complexing the molecule(s) with a composition to render it resistant to acidic and enzymatic hydrolysis, or by packaging the molecule(s) in an appropriately resistant carrier, such as a liposome or a protection barrier or by modifying the molecular size, weight, and/or charge of the modulator. Means of protecting agents from digestion degradation, and excretion are well known in the art.

The compositions for administration will commonly comprise a longevity modulator dissolved in a physiologically acceptable carrier, typically an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gilman, The Pharmacological Basis of Therapeutics (Hardman et al., eds., 1996)).

Thus, a typical pharmaceutical composition for intravenous administration would be about 0.1 to 10 mg per patient per day by weight. Dosages from 0.1 up to about 100 mg per patient per day (by weight) may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gilman, The Pharmacological Basis of Therapeutics, supra.

The compositions containing modulators can be administered for therapeutic or prophylactic treatments. In therapeutic applications, compositions are administered to a subject in an amount sufficient to cause some effect on the gene product of a biomarker of longevity. An amount adequate to accomplish this is defined as a “therapeutically effective dose.” Amounts effective for this use will depend upon the patient and factors such as the general state of the patient's health. Single or multiple administrations of the compositions may be administered depending on the dosage and frequency as required and tolerated by the patient. An amount of modulator that is capable of changing, e.g., preventing or slowing, the gene product profile characteristic of caloric restriction or dwarfism in a mammal is referred to as a “prophylactically effective dose.” The particular dose required for a prophylactic treatment will depend upon the medical condition and history of the marmnal, or particular type of complication being prevented, as well as other factors such as age, weight, gender, administration route, efficiency, etc.

It will be appreciated that the present longevity biomarker-modulating compounds can be administered alone or in combination with additional longevity interventions.

Kits

For use in diagnostic, research and therapeutic applications suggested above, kits are also provided by the invention. In the diagnostic and research applications such kits may include any or all of the following: assay reagents, buffers, longevity biomarker-specific nucleic acids or antibodies, hybridization probes and/or primers, small molecule modulators of longevity biomarkers, etc. A therapeutic product may include sterile saline or another pharmaceutically acceptable emulsion and suspension base. Kits for screening for modulators of the expression levels of longevity biomarkers are also provided.

The kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

A wide variety of kits and components can be prepared according to the present invention, depending upon the intended user of the kit and the particular needs of the user. For example, identification of candidate interventions would typically involve evaluation of a plurality of genes or products. The genes will be selected based on correlations described herein, which may be identified in historical data.

### EXAMPLES

The following examples are provided by way of illustration only and not by way of limitation. Those of skill in the art will readily recognize a variety of noncritical parameters that could be changed or modified to yield essentially similar results

To gain insight into the molecular pathways activated by DF and CR, analyzed gene expression profiles in the liver of normal (NL) and Ames DF mice subjected to ad libitum (AL) or CR diets using Affymetrix oligonucleotide microarrays containing probes for over 12,000 transcription units. The results, and the functional categories of genes affected by DF and CR, suggest that their additive enhancement of lifespan results from the greater number of genes affected by the combined treatments, and from additive effects on the expression on a subset of genes.

Materials and Methods

Mice. Male and female mice of the Ames stock were bred and housed at Southern Illinois University. DF (df/df) and NL (+/+ or +/df) mice were produced by crosses between df/+ parents or between fertile df/df males and df/+females (Bartke, et al., Exp. GerontoL 36:21-28, 2001). Details of the animal husbandry were as described. Mice had free access to tap water and standard pelleted food (LabDiet, PMI Feeds, Inc., St Louis, Mo.). The cages were equipped with microisolator filter tops. The room was maintained at 22±2° C. Lights were on from 0600-1800 h. Sentinel animals were negative for all pathogens tested.

Study Design. Starting at the age of 2 months, 16 female Ames DF and 16 of their NL littermates were randomly assigned to two dietary regimens. For each of the two genotypes, 8 mice were chosen randomly and subjected to CR while the remaining 8 continued AL feeding. This genotype/diet design resulted in 4 experimental groups: NL genotype AL fed (NLAL); NL genotype CR (NLCR); DF genotype AL (DFAL); and DF genotype CR (DFCR). The CR regimen was introduced progressively by reducing the daily food intake of CR mice to 90% of the AL intake of animals of the same genotype for 1 week, to 80% for the next week, and to 70% for the remainder of the study. Food consumption of the AL fed animals was monitored throughout the study, and the CR mice were fed daily, at approximately 1700 h, 70% of the average amount consumed daily by AL mice during the preceding week. Mice were killed at 6 months of age, tissues removed, rapidly frozen on dry ice, and stored in liquid nitrogen. The average weights of the mice at the end of the experiment were: NLAL, 30.1±4.5 g; NLCR, 23.6±1.6 g; DFAL, 15.4±2.3 g; DFCR, 10.4 ±0.6 g (SD).

Probe Set Expression Measurement and Normalization. Total liver RNA was isolated from frozen tissue as described (Dhahbi, et al., Diabetes Technol. Ther. 5:411-420, 2003). The mRNA levels were measured using the Affymetrix mouse U74Av2 array according to standard protocols (Dhahbi, et al., Diabetes Technol. Ther. 5:411-420, 2003; Cao, et al., Proc. Natl. Acad. Sci. U.S.A. 98:10630-10635, 2001). After hybridization, arrays were scanned using a Hewlett-Packard GeneArray Scanner. Image analysis was performed as described (Cao et al., Proc. Natl. Acad. Sci. U.S.A. 98:10630-10635, 2001). Raw image files were converted to probe set data (*.CEL files) using Microarray Suite (MAS 5.0). Probe set data from all 31 arrays were simultaneously analyzed with the Robust Multichip Average (RMA) method to generate normalized expression measures for each probe set (Irizarry, et al., Nucleic Acids Res. 31:e15, 2003). The data were further filtered to include only probe data sets that were “Present” in at least 75% of the arrays per experimental group according to the MAS 5.0 detection algorithm, which uses the Wilcoxon signed rank test (Wilcoxon, Biometrics 1:80-83, 1945; Affymetrix. (2001) Technical Notes 1, Part No. 701097 Rev. 1, 2001. Gene names were from the LocusLink and Affymetrix databases as of Nov. 19, 2003).

Data Analysis. We performed two-way analysis of variance (two-way ANOVA) in which expression level was considered to be a function of genotype only, diet only, or a function of genotype and diet. The two-way ANOVA test is based on the following model: yᵢⱼₖ=μ+Gᵢ+Dⱼ+(G×D)ᵢⱼ+εᵢⱼₖ where μ is the overall mean of log-transformed intensity values of gene expression that is common to all 31 samples; Gᵢ, is the effect of the i^{th} genotype (i=1, 2; 1=DF, 2=NL); Dⱼ is the effect of the j^{th} diet (j=1, 2; 1=CR, 2=AL); (G×D)ᵢⱼ is the interaction between genotype and diet; and εᵢⱼₖ is the stochastic error. An interaction between genotype and diet would indicate that the effect of CR on gene expression is conditional on the DF genotype. There are 8 replicates in each of the NLCR, DFAL, and DFCR sample sets (k=1, 2, . . . 8) and 7 replicates in the NLAL sample set (k=1, 2, . . . 7). Based on this model, yᵢⱼₖ represents the observed log-transformed intensity value of gene expression for the k^{th} replicate of the i^{th} genotype under j^{th} diet. The two-way ANOVA model was fitted to the sample data {yᵢⱼₖ} by the least square method.

The two-way ANOVA analysis csisisted of three statistical significance tests: a test of each of the two main effects (diet and genotype) and a test of the interaction between diet and genotype. We started by testing the hypothesis of no interaction. If the hypothesis of no interaction was rejected, we stopped further testing of the two main effects since such a statistically significant interaction indicates that diet and genotype effects are dependent on each other. If the hypothesis of no interaction was accepted, we continued the analysis by examining the effects of diet and genotype under the same two-way ANOVA model.

For each gene we calculated the F statistic using the LM procedure embodied in R to test the hypothesis of no interaction. This method assumes normality and homoscedasticity. Our statistical significance criterion for assessing the existence of interaction was the false discovery rate (<0.05) criterion. If the hypothesis of no interaction was accepted for a tested gene, the F statistics corresponding to each of the two main effects and the nominal P-values were calculated separately under the two-way ANOVA model. With a series of multiple simultaneous tests, the nominal P-values were adjusted to reduce the type I errors.

If a gene is upregulated (or downregulated) by CR only, the fold change of CR versus AL was estimated by 2^{|D}^{₁}^{-D}^{₂}^{|} (or −2^{|D}^{₁}^{-D}^{₂}^{|}). Similarly for a genotype only effect, the fold change of DF versus NL was estimated by 2^{|G}^{₁}^{-G}^{₂}^{|} (or −2^{|G}^{₁}^{-G}^{₂}^{|}). If a gene is upregulated (or downregulated) by both CR and DF independently, the fold change of DFCR versus ALNL was estimated by 2^{|D}^{₁}^{-D}^{₂}^{+G}^{₁}^{-G}^{₂}^{|} (or −2^{|D}^{₁}^{-D}^{₂}^{+G}^{₁}^{-G}^{₂}^{|}). When there is an interaction between the effects of diet and genotype, the fold change of DFCR versus ALNL was estimated by 2^{|D}^{₁}^{-D}^{₂}^{+G}^{₁}^{-G}^{₂}^{+(D×G)}^{₁₁}^{-(D×G)}^{₂₂}^{|} (or −2^{|D}^{₁}^{-D}^{₂}^{+G}^{₁}^{-G}^{₂}^{+(D×G)}^{₁₁}^{-(D×G)}^{₂₂}^{|}).

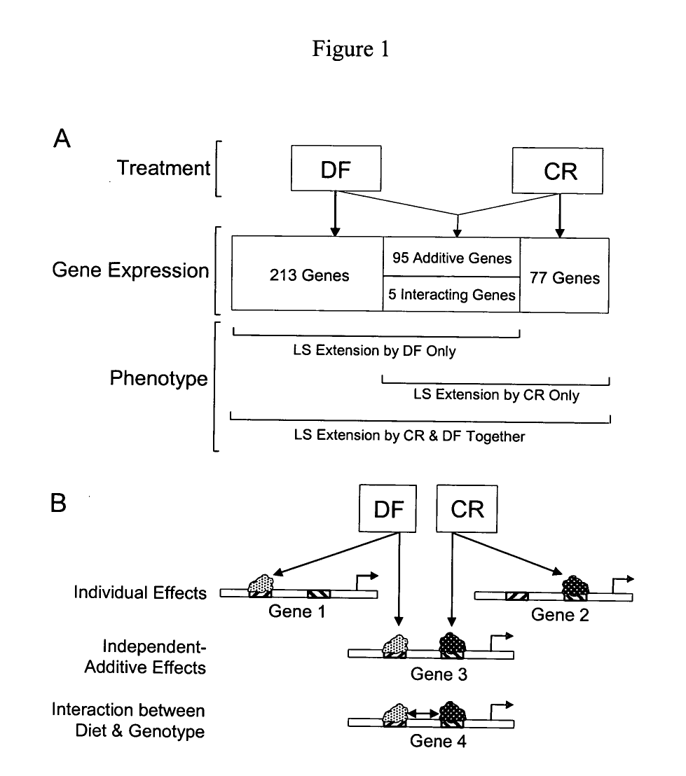

Four statistical categories of genes changed by DF, CR or both interventions were identified by two-way ANOVA, based on the model, yᵢⱼₖ=μ+Gᵢ+Dⱼ+(G×D)ᵢⱼ+εᵢⱼₖ, described in Supporting Materials and Methods (FIG. 1A). These groups were: 213 genes affected only by DF (Gᵢ≠0, Dⱼ=0, (G×D)ⱼ=0); 77 genes affected only by CR (Dⱼ≠0, Gᵢ=0, (G×D)ⱼ=0); 95 genes affected additively but independently by both interventions (Dⱼ≠0, Gᵢ≠0, (G×D)ⱼ=0); and 5 genes for which the effects of diet were dependent on genotype (Dⱼ≠0, Gᵢ≠0, (G×D)ⱼ≠0), where G is genotype, D is diet, and GxD is interaction between diet and genotype.

Validation of Microarray Results. The expression of a total of 16 genes was examined by qPCR (Rajeevan, et al., J. Mol. Diagn. 3:26-31, 2001). Real-time, two-step RT-PCR was performed with a QuantiTect SYBR Green PCR kit (Qiagen, Hilden, Germany) and an ABI PRISM 7700 Sequence Detection System (Applied Biosystems, Foster City, Calif.). Primers were designed using the Netaffx analysis center and PCR products sequenced and verified against the public database (Table 1). Primers for transcription elongation factor A 1 (SII) were amplified in parallel with the genes of interest as a control. SII mRNA is unaffected by CR and DF (data not shown). Amplification specificity was confirmed by melting curve analysis and agarose gel electrophoresis using standard techniques.

TABLE 1
Primer sequences for qPCR

| | | | |
|---|---|---|---|
| | | Primer sequences (5′-3′) | Product |
| Gene Name | GenBank | (Forward and Reverse Primers) | size (bp) |

| | | | |
|---|---|---|---|
| CCAAT/enhancer binding | X61800 | CAGTTCTTCAAAAAACTGCCCAGC | 153 |
| protein, delta | | AAAGAAACTAGCGATTCGGGCG | |
| Cell line NK14 derived | AI842492 | TGATTTTCTAGCAGCATACCTGGGA | 135 |
| transforming oncogene | | ATCACAACTGGGTAAAGACAGCAGG | |
| Cytochrome P450, 4a14 | Y11638 | TTGGGCCAAACTGTGAAAAAATC | 118 |
| | | ATTGCCAAAACTGCTCTGGCTC | |
| Cytochrome P450, 2f2 | M77497 | GCTTCCTCACAAAGATGGCACAG | 106 |
| | | GTTTCTGTGCCACCGAAGAGC | |
| Fatty acid synthase | X13135 | TTGGGTTTTGACTTTTCTGCAGCTG | 123 |
| | | CACGTGCAGTTTAATTGTGGGATCA | |
| G0/G1 switch gene 2 | X95280 | CAGAGCTCAGATGGAAAGTGTGCAG | 152 |
| Phenylalanine | | TGCACACCGTCTCAACTAGGCC | |
| Glyoxalase 1 | AI852001 | GGTCTGTTACCTTCTGGGGTTTCAG | 158 |
| | | TGATTCCGAATTGCTCTCAGGAGTA | |
| Insulin-like growth factor 1 | X04480 | CACGGAGCAGAAAATGCCACA | 129 |
| | | CATTGGGGGAAATGCCCATC | |
| Insulin-like growth factor | X81580 | AGTGCTGGTGTGTGAACCCCAATAC | 107 |
| binding protein 2 | | ACCAGTCTCCTGCTGCTCGTTGTAG | |
| Long-chain fatty-acyl | AI839004 | CATCGTCCCTGGAGCTGAACAG | 119 |
| elongase | | CCAGGATTATGTGTGAGGTCGAACA | |
| Metallothionein 1 | V00835 | CTCCTGCGCCTGCAAGAACTG | 96 |
| | | ACACAGCCCTGGGCACATTTG | |
| p300/CBP-associated factor | AW047728 | GCTTCTGACATGGAAGGCATG | 157 |
| | | ACCAGTCTGAGACACTTAATGCAGC | |
| Peroxisome proliferator | X57638 | CAGTCCCCAGTCTGGTCTTAACCG | 120 |
| activated receptor alpha | | GGAAGGGAACAGACCGCTCAGAC | |
| Quiescin Q6 | AW045751 | TCAGTGCTCTACTCGTCCTCTGACC | 115 |
| | | CACACCAGGAGGCGAAGAACTC | |
| Thyroid hormone responsive | X95279 | CCACCTCTGGGATGTCGTTTAGTGC | 121 |
| SPOT14 homolog (Rattus) | | AGGGCTTTGGATTCCGTGTTTG | |
| Transcription elongation | M18209 | CCAGCTGAAATGTAGGCTGTAGCAA | 199 |
| factor A (SII) 1 | | ACAGGAGTCTGAACACAGGCAGAAG | |
| U2 small nuclear | X64587 | TTCCCCCATGGTAGGAACATAGC | 140 |
| ribonucleoprotein auxiliary | | AGAACAGGAAGGACCAGAAGCCA | |
| factor (U2AF), 65 kDa | | | |

Results and Discussion

Data Analysis. Four statistical categories of genes changed by DF, CR or both interventions were identified by two-way ANOVA (FIG. 1A). These groups were: 213 genes affected only by DF; 77 genes affected only by CR; 95 genes affected additively but independently by both interventions; and 5 genes for which the effects of diet were dependent on genotype. Since only 5 of 390 changed genes were conditional on genotype, CR and DF work largely independently to regulate gene expression. However, this does not necessarily imply that CR and DF work through completely independent pathways. CR and DF might independently and additively regulate gene expression by changing the activity of discrete transcription factors (FIG. 1B). Alternatively, co-regulation could be mediated by cross-talk between signal transduction systems or effects on different steps in gene expression.

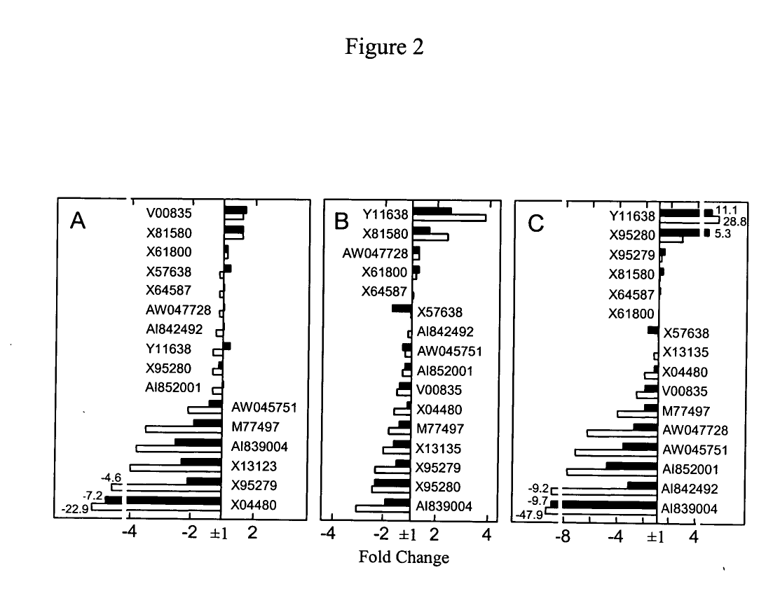

Validation by qPCR. To insure the analysis resulted in a low false discovery rate, the expression of 16 randomly chosen genes was reanalyzed using qPCR (FIG. 2). Changes in the expression of all 16 genes were verified as to direction and magnitude. Thus, the methods used are reliable. In general, qPCR found a greater change in gene expression than was found by microarrays (FIG. 2).

Functional Classification of Genes. To explore the effects of DF and CR, we functionally classified the changed genes (Table 2). A complete list of changed genes is given in Table 3. DF and CR alone and in combination had major effects on genes associated with energy metabolism (18, 18, and 11%), transcription (10, 7 and 4%), signal transduction (10, 8 and 11%), and xenobiotic and oxidant metabolism (5, 5, and 11%).

Gluconeogenesis. Separately and in combination, DF and CR enhanced gene expression associated with gluconeogenesis (Table 2; Pck1, Glu1, Gpi1 and G6pt1). Individually, and additively in combination, they enhanced expression of the key gating enzyme of gluconeogenesis, Pck1. DF decreased Glu1 expression, which may reduce the rate of glutamine synthesis in the liver, sparing glutamate for gluconeogenesis. CR upregulated Gpi1 and G6pt1, genes important for gluconeogenesis. These results and our previous studies of CR (Cao et al., Proc. Natl. Acad. Sci. U.S.A. 98z′10630-10635, 2001; Dhahbi, et al., Am. J. Physiol. 277:E352-E360, 1999), suggest that DF and CR individually enhance the enzymatic capacity for the turnover and renewal of hepatic and extrahepatic protein, and these effects are additively enhanced in DFCR mice.

Glycolysis. Several key enzymes involved in liver glycolysis were underexpressed in DF mice (Gck and Pklr). It has previously been shown that CR decreases glucokinase, pyruvate kinase and acetyl CoA carboxykinase expression (Dhahbi, et al., Mech. Ageing Dev. 122:35-50, 2001). Together these results suggest that DF and CR decrease substrate availability for de novo lipogenesis in the liver.

Lipid and Cholesterol Metabolism. DF and CR decreased the expression of genes key to hepatic lipogenesis (Table 2). Hepatic expression of 16 lipid- and cholesterol-related genes were underexpressed in DF mice. These genes are involved in lipid transport (Apoa4, Pltp, Plscr2, Cte1, Fabp2 and Dbi) and uptake (Mgll), fatty acid synthesis (Acly, Mod1, Fasn and Thrsp) and cholesterol biosynthesis (Fdps, Sqle, Cyp51, Nsdhl and Dhcr7). DF and CR alone, and additively in combination downregulated Lipc, an important enzyme in HDL metabolism, Elov16, a key enzyme of lipid synthesis, and Ebp, a key enzyme of cholesterol synthesis (Table 2). The expression of Apoc2 was additively upregulated by DF and CR. The product of this gene is a potent activator of lipoprotein lipase, and plays an important role in the catabolism of triglyceride-rich lipoproteins. DF and CR individually and additively together enhanced the expression of key enzymes involved in β-oxidation of fatty acids [Hadh2 and Cyp4a14 (by 11.1-fold)]. Separately, DF and CR upregulated the expression of Amacr Acadm, Cyp4a10, Peci, Hadhb, and Cpt1a. Taken together, these results suggest that DF and CR individually and additively enhance the enzymatic capacity for gluconeogenesis and lipid utilization for energy production, and suppress the capacity for glycolysis and de novo lipogenesis.

Eight of the 16 lipid-related genes are transcriptionally regulated by sterol response element binding proteins (SREBPs; Table 2; Refs (Foufelle & Ferre Biochem. J. 366:377-391, 2002; Horton et al., J. Clin. Invest 109:1125-1131, 2002). Thus, disrupted GH/IGF1 signaling in DF mice may reduce lipid and cholesterol metabolism by modulating the activity of transcription factor SREBPs. A similar mechanism has been proposed to explain the underexpression of fatty acid- and cholesterol synthesis-related genes in the liver of hypophysectomized rats (Frick, et al., Am. J. Physiol Endocrinol. Metab 283:E1023-E1031, 2002).

Young adult dwarf mice have more body fat than normals. But, with age normal mice from this line accumulate fat at a higher rate, and the percent body fat in old DF mice does not differ from that of normals, as measured by DEXA (Heiman, et al., Endocrine 20:149-154, 2003). Downregulation of lipid biosynthetic genes and upregulation of β-oxidation related genes in the liver of DF mice may explain this slower rate of fat deposition.

IGF1-phosphatidylinositol 3-kinase (PI3K)-Forkhead Transcription Factor Cascade. The most likely source of the longevity effects of DF is the suppression of GH/IGF1 signaling (Longo & Finch Science 299:1342-1346, 2003). Suppression of GH production reduces IGF 1 synthesis in the liver, which reduces the activity of PI3K. This results in upregulation of forkhead transcription factors and thereby upregulation of genes coding for stress-resistance, including anti-oxidant enzymes. CR also reduced GH/IGR-1 signaling and downregulated Pik3r1 expression. However, the importance of these effects in the additive lifespan effects of DF and CR is unclear. DF alone reduces GH/IGF1 signaling by 90%. Thus, the additive effects of DF and CR on lifespan must involve at least one other signaling pathway.

In this regard, the forkhead transcription factors Foxa2 and Foxa3 were underexpressed and overexpressed, respectively, in CR and DF mice, and the interventions additively regulated these genes in DFCR mice (Table 2). Foxa-binding sites exist upstream of more than 100 genes that are expressed in the liver, pancreas, intestine, and lung (Kaestner Trends Endocrinol. Metab 11:281-285, 2000). Foxa isoforms regulate liver genes including phosphoenolpyruvate carboxykinase (PEPCK; Pck1), glucose-6-phospatase, fructose-2,6-bisphosphatase, catalase and IGF-binding protein 1 (Igfbp1; Kaestner, et al., Mol. Cell Biol. 18:4245-4251, 1998; Shen, et al., J. Biol. Chem. 276:42812-42817, 2001). Overexpression of Foxa2 is associated with steatosis and mitochondrial damage (Hughes, et al. Hepatology 37:1414-1424, 2003). Foxa3 is central to the maintenance of differentiated functions in hepatocytes and is a homolog of Daf-16, a forkhead transcription factor which regulates lifespan in C. elegans (Lin, et al. Science 278:1319-1322, 1997). Foxa3 may regulate glucose homeostasis through control of PEPCK, transferrin, tyrosine aminotransferase, and glucose transport protein 2 expression (Kaestner Trends Endocrinol. Metab 11:281-285, 2000; Kaestner, et al., Mol. Cell Biol. 18:4245-4251, 1998; Shen, et al., J. Biol. Chem. 276:42812-42817, 2001). Foxa3 may enhance stress resistance through induction of catalase and the repression of cell proliferation (Nakamura et al., Biochem. Biophys. Res. Commun. 253:352-357, 1998). Thus, the additive switch from Foxa2 to Foxa3 expression in DFCR mice may lead to the additive induction of gluconeogenesis and stress resistance and reduced cell proliferation. These effects may be key to the additive effects of DF and CR on lifespan.

Insulin sensitivity. DF and CR caused underexpression of Gas6, a growth factor ligand for the Axl tyrosine kinase receptor. Axl interacts with the product of the Pten gene, and reduced Pten signaling improves insulin sensitivity and normalizes glucose concentration in genetically diabetic mice. Thus, reduced Gas6 and Pten signaling may result in the enhancement of insulin sensitivity in DF and CR mice (Dhahbi, et al., Mech. Ageing Dev. 122:35-50, 2001; Dominici, et al., J. Endocrinol. 173:81-94, 2002). Ames dwarf mice are known to have increased insulin receptor content, phosphorylation of IRS-1 and -2, association of the p85 regulatory subunit of PI3K with IRS-1, and enhanced activation of insulin-stimulated protein kinase B (Dominici, et al., J. Endocrinol. 173:81-94, 2002).

Glucagon and epinephrine sensitivity. DF upregulated adcy6 and adcy9 (Table 2). These plasma membrane bound-proteins catalyze the formation of cAMP in hepatocytes. Adcy6 is activated by forskolin and glucagon while Adcy9 is stimulated by β-adrenergic receptor agonists but is insensitive to Ca(2+)/calmodulin, forskolin and somatostatin. Upregulation of these enzymes should enhance hepatic sensitivity to glucagon and epinephrine, and increase glycogenolysis and glucose output during fasting.

Carcinogenesis in DF and CR rodents. The DF mutations reduce the incidence and growth of spontaneous and transplanted tumors in mice (Ikeno, et al., J. GerontoL A Biol. Sci. Med. Sci. 58:291-296, 2003). Igf1, which is negatively regulated by DF, is a key regulator of mitogenesis and tumorigenicity, and plays a crucial role in the survival of transformed cells in vivo (Rubini, et al., Exp. Cell Res. 251:22-32, 1999). In tumor cells, IGF1 acts as an autocrine/paracrine growth factor as well as an inhibitor of apoptosis. Defects in IGF 1 receptor expression and/or activation inhibit tumorigenicity, reverse the transformed phenotype, and cause massive apoptosis in vitro and in vivo (Rubini, et al., Exp. Cell Res. 251:22-32, 1999; Burfeind, et al., Proc. Natl. Acad. Sci. U.S. A 93:7263-7268, 1996). CR also has a well described anti-carcinogenic effect on spontaneous and chemically induced tumors (Hursting, et al., Annu. Rev. Med. 54:131-52, 2003). Reduction of cell proliferation and induction of apoptosis are thought to be the mechanisms for these effects of CR in liver. In addition, downregulation of Fasn and the fatty-acid-synthesis pathway by DF and CR may have anticancer effects (Table 2; see, also, Cao, et al, Proc. Natl. Acad. Sci. U.S. A. 98:10630-10635, 2001). Both genes are required for the survival of many human cancer cell lines, and inhibition of Fasn leads to apoptosis in cancer cells (Kuhajda, et al., Proc. Natl. Acad. Sci. U.S. A 97:3450-3454, 2000; Pizer, et al., Cancer Res. 58:4611-4615, 1998).

Cell proliferation. As expected, DF alone (−7.2-fold) and in combination with CR (−9.7-fold) strongly downregulated Igf1 mRNA. Consistent with these observations, DF repressed the expression of Mup1, Mup3, Mup4, and Mup5, which are repressed by low GH levels (Johnson, et al., J. Mol. Endocrinol. 14:21-34, 1005). CR has a similar effect on the expression of these genes, consistent with the 70% reduction in serum IGF1 levels in CR mice (Cao, et al, Proc. Natl. Acad. Sci. U.S.A. 98:10630-10635, 2001, Gat-Yablonski, et al, Endocrinology 145:343-350, 2004). DF and CR also additively induced the expression of Igfbp2 by 3-fold, and DF upregulated Igfbp1 7.2-fold. Previously, it was found that CR induces the expression of IGF binding protein 7 (Cao, et al, Proc. Natl. Acad. Sci. U.S.A. 98:10630-10635, 2001). IGF binding proteins are generally regarded as inhibitors of the growth promoting effects of the IGFs, suggesting that both DF and CR strongly inhibit IGFI signaling.

DF and CR additively induced expression of cellular repressor of Creg, an inhibitor of cell growth (Veal, et al., Mol. Cell Biol. 18:5032-5041, 1998). DF down-regulated suppressor of Socs2, which is part of a classical negative feedback system that down-regulates GH/IGF1 signaling. It may be underexpressed in response to the reduced GH/IGF1 signaling in DF mice.

DF and CR produced changes in gene expression consistent with reduced cellular growth and cellular stress. The Prlr was negatively regulated by 3.0- and 1.3-fold in DF and CR mice, and in DFCR mice the receptor mRNA was reduced by 4.5-fold. This downregulation should exacerbate the already reduced prolactin signaling in DF mice. The role of prolactin in the liver is unclear, but it induces hepatic hypertrophy, and may regulate hepatocyte renewal. Likewise, DF and CR led to substantial downregulation of Lifr. This cytokine receptor affects the proliferation of a wide variety of cells, and this system affects other signaling systems, including those for GH and prolactin. Mig6/Gene 33, an adapter protein that is induced by diabetes and persistent stress was downregulated in DF and DFCR mice. DF mice underexpressed Ccndl, Pole4, Cetn 2 (which is essential for centriole duplication), Nrp, and Serpina3c (which may be involved in inflammation and cell growth). DF resulted in overexpression of Tgfbi, a putative mediator of the growth inhibitory effects of TGFβ. CR downregulated GOs2, which is upregulated following receipt of mitogenic stimuli, and upregulated Tieg1, a putative tumor suppressor-like transcriptional repressor, and Prkcn/pkd3, a diacylglycerol responsive, serine-threonine kinase which activates mitogen-activated protein kinase. DF also suppressed the expression of Shmt1, which generates single carbon units for purine, thymidine, and methionine biosynthesis.

Apoptosis. Hepatocytes from DF mice have enhanced rates of apoptosis in response to oxidative insult (Kennedy, et al., Exp. Gerontol. 38:997-1008, 2003). Short- and long-term reductions in caloric intake are correlated with increased programmed cell death (Hursting, et al., Annu. Rev. Med. 54:131-52, 2003). Two mechanisms may be responsible for the effects of CR on apoptosis, reduced IGF1 signaling and reduced endoplasmic reticulum (ER) chaperone gene expression. Globally active, circulating factors, especially IGF1, are thought to regulate mitogenic signaling and apoptosis in many types of normal and cancer cells, including hepatocytes (Hursting, et al., Annu. Rev. Med. 54:131-52, 2003, Dunn, et al., Cancer Res. 57:4667-4672, 1997). The additive underexpression of IGF1 induced by DF and CR may produce additive suppression of cell proliferation and additively tip the molecular balance toward apoptosis in liver via a variety of downstream genes. In agreement with this hypothesis, DF, alone and in combination with CR induced a pattern of gene expression consistent with increased apoptotic potential (Table 2). DF and CR additively induced the expression of the apoptosis-mediator Casp6, and repressed the expression of Psen2, the familial Alzheimer's disease gene. Psen2 is both required for apoptosis, and is processed by caspase 3 into an anti-apoptotic COOH-terminal polypeptide that antagonizes the progression of cell death (Vito, et al., J. Biol. Chem. 272:28315-28320, 1997). DF induced the expression of Gas2, which is highly expressed in growth-arrested cells and induces rearrangement of the actin cytoskeleton during apoptosis (Benetti, et al., EMBO J. 20:2702-2714, 2001). DF led to underexpression of Rgn (SMP30), which protects cells from apoptosis (Ishigami, et al., Am. J. Pathol. 161:1273-1281, 2002). CR induced overexpression of Tieg1, which can induce apoptosis in a pancreas-derived cell line, as can TGFβ (Ribeiro, et al., Hepatology 30:1490-1497, 1999).

DF alone and additively in combination with CR decreased the expression of 8 chaperone genes (Table 2). We have previously shown that the mRNA and protein levels of most hepatic endoplasmic-reticulum chaperones increase with age and decrease with CR and fasting, most likely in response to changes in the insulin to glucagon ratio (Dhahbi, et al., J. Nutr. 132:31-37, 2002). Reduced chaperone expression increases apoptotic responsiveness to genotoxic stress through both the endoplasmic stress and the mitochondrial apoptosis signaling pathways (Suh, et al., Nat. Med. 8:3-4, 2002; Rao, et al., FEBS Lett. 514:122-128, 2002). Thus, DF, CR and fasting reduce ER chaperone levels, and thereby enhance apoptosis in liver, perhaps accounting for their anti-cancer benefits (Grasl-Kraupp, et al., Proc. Natl. Acad. Sci. U.S.A. 91:9995-9999, 1994; Jamora, et al, Proc. Natl. Acad. Sci. U.S.A. 93:7690-7694, 1996).

In contrast to the results above, DF and CR upregulated Hspa9a, Hspalb, and Herpud1. Hspa9a and hspalb, homologues of the hsp70 family which differ by only 2 amino acids were induced by CR in NL and DF mice. Hspa9a (mortalin-1), is antiproliferative in normal cells and may be a chaperone in mitochondria and the ER. Hspa1b (mortalin-2) has proliferative functions, can repress p53-mediated transcriptional transactivation via a nuclear-exclusion mechanism, and may be a chaperone involved in intracellular trafficking and mitochondrial import. Herpud1 is an ER resident chaperone thought to regulate ER-associated protein degradation. It also represses transcription as a heterodimer with other factors. Thus, induction of these multifunctional chaperones may contribute to the repressive molecular environment for cell growth in DF and CR liver.

Oxidant and Toxin Defense. Oxidative and other genotoxic damage to DNA has been implicated in tumor formation (Cooke, et al., FASEB J. 17:1195-1214, 2003). We found additive induction of 8 phase I and II xenobiotic metabolism-related genes by DF and CR (Cyp2d9, Cyp3a16, Fmo5, Ephx1, Ephx2, Gstm1, Gstm3, and Gstp2). In addition, DF upregulated 3 such genes (Cyp3a25, Gsta2, and Gsta4), and CR upregulated 2 of these genes (Cyb5r1-pending and Gstt2). We also found that DF and CR alone and in combination upregulated Gclc expression, a rate-limiting enzyme in the synthesis of glutathione, which plays a crucial role in the intracellular antioxidant defense systems. In addition, DF upregulated Gpx4 and Tdpx-ps1, and CR upregulated Gsr expression. The function of this gene is unknown at present. The upregulation of genes for xenobiotic and antioxidant metabolism might enhance lifespan through their anti-carcinogenic effects (Sheweita & Tilmisany Curr. Drug Metab 4:45-58, 2003). Interestingly, Es31, a carboxylesterase with uncharacterized substrate specificity was 5- to 6-fold downregulated by DF.

DF induced three members of the ATP-binding cassette membrane multidrug resistance transporters (Abcc3, Abcc2 and Abcg2). In liver, Abcc3, multidrug resistance transporter 3, exports a wide range of organic anions back to the blood, thereby decreasing exposure and toxicity to the liver. Abcc2 mediates ATP-dependent transport of various amphipathic endogenous and xenobiotic compounds across the canalicular membrane into bile, and is a major driving force for bile flow. Abcg2, which codes for a transmembrane transporter localized in the liver bile canaliculi protects the organism from potentially harmful xenobiotics. These results suggest that DF mice have enhanced protection from potentially harmful endogenous and xenobiotic toxins.

DF induced intracellular solute carrier transporters for cationic amino acids (Slc7a2); vitamin C (Slc23a1); many monocarboxylates, such as lactate, pyruvate, branched-chain oxo acids derived from leucine, valine and isoleucine, and α-ketoacids (Slc16a7). Together these data are consistent with the evidence for enhanced gluconeogenic activity and protein turnover found in DF mice discussed above.

DF repressed the expression of Slc10a1, which encodes a hepatocyte specific transporter for the uptake of taurocholate and other bile salts; Slc22a1, which encodes the main receptor for uptake of a variety of structurally diverse organic cations and toxins in hepatocytes; and Slc29a1, an equilibrative nucleoside transporter that plays an important role in adenosine-mediated regulation of physiological processes and the uptake of cytotoxic nucleosides. Dwnregulation of these genes should decrease the uptake of potentially toxic xenobiotics and endogenous substances.

Published studies of DF gene expression. Results were compared to the limited cDNA array gene expression studies in the literature. A previous study found 3 changed genes in the liver of Ames DF mice, and our results confirm 2 of these changes (Igfbp2 and Igfla; (Dozmorov, et al., J. Gerontol. A Biol. Sci. Med. Sci. 56:B72-B80, 2001). These authors also found 17 “rigorously significant” changes in the liver of Snell DF mice. Our studies confirm 3 of these changes (Igf1, Igfbp2, Mup1; Ref. (Dozmorov, et al., J. Gerontol. A Biol. Sci. Med. Sci. 57:B99-108, 2002). A study of GH receptor knockout (GHR-KO) mice, which also have the DF phenotype, found no changed genes that met their statistical criteria for significance, and no evidence for overlapping effects of DF and CR (Miller, et al., Mol. Endocrinol. 16:2657-2666, 2002). The differences with our data are likely due to the larger number of gene-expression probes in our studies, and differences in analytical and statistical methods.

The data described herein indicate that the majority of the effects of DF and CR on gene expression fall into two general categories. The first category of genes changed expression in response to only one intervention. The other category of genes was additively affected by the combination of the interventions. The genes in each of these categories were spread throughout the functional categories of genes affected by the interventions (Table 3). For example, the number of DF-, CR- and additively DFCR-responsive genes in the xenobiotic and oxidant metabolism, signal transduction, transcription and chaperone functional categories was approximately proportional to the total number of the DF-, CR- and additively DFCR-responsive genes (FIG. 1A and Table 3). In contrast, the DF responsive genes were dominant in the categories of nucleotide metabolism, glycolysis, fatty acid synthesis, lipid transport, cholesterol synthesis, and immune system. Together, these results suggest that genes which are additively and individually affected by the interventions contribute to the additive effects of DF and CR on lifespan.

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE 2
Representative list of the effects of DF, CR and
DF and CR together on hepatic gene expression

| | | | | |
|---|---|---|---|---|
| Category/ | | | | |
| GenBank | Gene Symbol | DF | CR | DFCR |

Energy Metabolism
Glycolysis

| | | | | |
|---|---|---|---|---|
| L41631 | Gck | −1.3 | −1.1 | −1.5 |
| D63764 | Pklr | −1.5 | −1.2 | −1.9 |

Gluconeogenesis Related

| | | | | |
|---|---|---|---|---|
| AF009605 | Pck1 | 1.3 | 1.2 | 1.6 |
| U09114 | Glul | −1.4 | 1.0 | −1.4 |
| M14220 | Gpi1 | 1.1 | 1.2 | 1.4 |
| AF080469 | G6pt1 | 1.0 | 1.3 | 1.3 |

Lipid Uptake

| | | | | |
|---|---|---|---|---|
| Z22216 | Apoc2 | 1.4 | 1.3 | 1.8 |
| X58426 | Lipc | −1.6 | −1.2 | −2.1 |
| AI846600 | Mgll | −1.4 | −1.1 | −1.6 |

Lipid Transport

| | | | | |
|---|---|---|---|---|
| M64248 | Apoa4 | −4.3 | 1.3 | −3.6 |
| U28960 | Pltp | −1.7 | 1.0 | −1.7 |
| AF015790 | Plscr2 | −1.4 | 1.0 | −1.4 |
| Y14004 | Cte1 | −1.4 | 1.3 | −1.1 |
| M65034 | Fabp2 | −1.8 | −1.1 | −2.0 |
| X61431 | Dbi | −1.2 | −1.1 | −1.4 |

Fatty Acid Synthesis (Lipogenesis)

| | | | | |
|---|---|---|---|---|
| AI839004 | Elovl6 | −2.5 | −2.3 | −4.8 |
| AW121639 | Acly | −1.7 | −1.2 | −2 |
| J02652 | Mod1 | −2.0 | −1.1 | −2.3 |
| X13135 | Fasn | −2.3 | −1.6 | −3.6 |
| X95279 | Thrsp | −2.1 | −1.5 | −3.2 |

Cholesterol Synthesis

| | | | | |
|---|---|---|---|---|
| X97755 | Ebp | −1.4 | −1.2 | −1.6 |
| AW045533 | Fdps | −1.9 | −1.2 | −2.3 |
| D42048 | Sqle | −1.6 | −1.2 | −2 |
| AW122260 | Cyp51 | −1.6 | 1.0 | −1.6 |
| AW106745 | Nsdhl | −1.7 | −1.2 | −2.2 |
| AF057368 | Dhcr7 | −1.6 | 1.0 | −1.7 |

Beta-oxidation

| | | | | |
|---|---|---|---|---|
| U96116 | Hadh2 | 1.2 | 1.2 | 1.5 |
| Y11638 | Cyp4a14* | 1.2 | 2.5 | 11.1 |
| U89906 | Amacr | 1.2 | 1.2 | 1.4 |
| U07159 | Acadm | 1.2 | 1.1 | 1.4 |
| AB018421 | Cyp4a10 | 1.1 | 1.8 | 2.4 |
| AI840013 | Peci | 1.1 | 1.3 | 1.5 |
| AW122615 | Hadhb | −1.1 | 1.3 | 1.2 |
| AF017175 | Cpt1a | 1.1 | 1.3 | 1.4 |

Xenobiotic and Oxidant Metabolism
Phase I

| | | | | |
|---|---|---|---|---|
| M27168 | Cyp2d9 | 1.5 | 1.1 | 1.6 |
| D26137 | Cyp3a16 | 1.3 | 1.2 | 1.6 |
| U90535 | Fmo5 | 1.3 | 1.3 | 1.7 |
| Y11995 | Cyp3a25 | 1.6 | 1.2 | 1.9 |
| AI839690 | Cyb5r1- | 1.1 | 1.3 | 1.4 |
| | pending | | | |

Phase II

| | | | | |
|---|---|---|---|---|
| U89491 | Ephx1 | 1.2 | 1.2 | 1.5 |
| Z37107 | Ephx2 | 1.3 | 1.2 | 1.6 |
| J03952 | Gstm1 | 1.2 | 1.1 | 1.4 |
| J03953 | Gstm3 | 1.5 | 1.3 | 2.0 |
| X53451 | Gstp2 | 1.6 | 1.4 | 2.6 |
| J03958 | Gsta2 | 2.3 | 1.1 | 2.8 |
| L06047 | Gsta4 | 1.5 | 1.1 | 1.8 |
| X98056 | Gstt2 | 1.1 | 1.3 | 1.5 |

Anti-oxidant

| | | | | |
|---|---|---|---|---|
| U85414 | Gclc | 1.2 | 1.2 | 1.6 |
| D87896 | Gpx4 | 1.2 | 1.1 | 1.4 |
| AF032714 | Tdpx-ps1 | 1.2 | 1.1 | 1.4 |
| AI851983 | Gsr | 1.1 | 1.2 | 1.4 |

Others

| | | | | |
|---|---|---|---|---|
| L11333 | Es31 | −5.3 | −1.3 | −6.4 |

Signal Transduction
Growth Related

| | | | | |
|---|---|---|---|---|
| M22957 | Prlr | −3.0 | −1.3 | −4.5 |
| X04480 | Igf1 | −7.2 | −1.1 | −9.7 |
| X81580 | Igfbp2 | 1.6 | 1.7 | 3.0 |
| D17444 | Lifr* | −3.5 | −1.7 | −5.0 |
| AI853531 | Mig6* | −1.6 | 1.2 | −1.3 |
| U88327 | Socs2 | −2.7 | 1.0 | −2.8 |
| X81579 | Igfbp1 | 7.2 | 1.5 | 11.6 |
| D50086 | Nrp | −1.4 | −1.1 | −1.6 |
| X61597 | Serpina3c | −1.6 | 1.1 | −1.5 |
| L19932 | Tgfbi | 1.5 | 1.1 | 1.6 |
| U92437 | Pten | 1.1 | −1.7 | −1.6 |
| U50413 | Pik3r1 | −1.1 | −1.6 | −1.7 |
| AW124627 | Prkcn | 1.1 | 1.2 | 1.4 |

Cytokine and Others

| | | | | |
|---|---|---|---|---|
| M93422 | Adcy6 | 1.3 | 1.1 | 1.6 |
| Z50190 | Adcy9 | 1.3 | 1.1 | 1.4 |

Transcription

| | | | | |
|---|---|---|---|---|
| L10409 | Foxa2 | −1.2 | −1.2 | −1.5 |
| X74938 | Foxa3 | 1.2 | 1.3 | 1.6 |
| AF084524 | Creg | 1.2 | 1.2 | 1.5 |

Transport and Trafficking
Membrane
Transport

| | | | | |
|---|---|---|---|---|
| AI173996 | Abcc2 | 1.4 | 1.2 | 1.7 |
| AA833514 | Abcc3 | 1.3 | 1.1 | 1.4 |
| AF103875 | Abcg2 | 1.8 | 1.3 | 2.3 |
| Intracellular | | | | |

Transport

| | | | | |
|---|---|---|---|---|
| L03290 | Slc7a2 | 1.3 | 1.1 | 1.5 |
| U95132 | Slc10a1 | −1.4 | 1.1 | −1.2 |
| AF058054 | Slc16a7 | 1.5 | 1.1 | 1.7 |
| U38652 | Slc22a1 | −1.6 | −1.2 | −2.0 |
| AI844736 | Slc23a1 | 1.4 | 1.1 | 1.6 |
| AI838274 | Slc29a1 | −1.3 | 1.0 | −1.4 |

Cell Proliferation (Cell Cycle and DNA Replication)

| | | | | |
|---|---|---|---|---|
| X59846 | Gas6* | −1.5 | −1.4 | −2.0 |
| AI849928 | Ccnd1 | −1.6 | 1.2 | −1.3 |
| AW060791 | Pole4 | −1.3 | −1.1 | −1.5 |
| AL021127 | Cetn2 | −1.7 | −1.1 | −2.1 |
| AA913994 | Shmt1 | −1.4 | −1.2 | −1.7 |
| AF064088 | Tieg1 | −1.3 | 1.4 | 1.1 |
| X95280 | G0s2 | −1.1 | −1.9 | −2.0 |

Apoptosis

| | | | | |
|---|---|---|---|---|
| Y13087 | Casp6 | 1.2 | 1.2 | 1.4 |
| U57325 | Psen2 | −1.3 | −1.3 | −1.6 |
| M21828 | Gas2 | 1.7 | 1.0 | 1.8 |
| U32170 | Rgn | −1.2 | −1.1 | −1.4 |

Chaperone (Protein Folding)

| | | | | |
|---|---|---|---|---|
| AI846938 | Herpud1 | 1.2 | 1.3 | 1.6 |
| AF055664 | Dnaja1 | −1.2 | −1.2 | −1.5 |
| AA615831 | Hspa4 | −1.4 | −1.2 | −1.7 |
| L40406 | Hsp105 | −1.6 | −1.1 | −1.7 |
| J04633 | Hspca | −1.5 | −1.1 | −1.7 |
| AW122022 | Ppid | −1.2 | −1.2 | −1.4 |
| AI842377 | P5-pending | −1.4 | 1.0 | −1.4 |
| AV373612 | Bag3 | −1.3 | 1.0 | −1.4 |
| D17666 | Hspa9a | 1.1 | 1.3 | 1.4 |
| AF109906 | Hspa1b | 1.1 | 1.5 | 1.8 |
| AA879709 | Ssr1 | −1.1 | −1.3 | −1.5 |

Pheromone

| | | | | |
|---|---|---|---|---|
| M17818 | Mup1 | −4.6 | −1.2 | −7.4 |
| M16357 | Mup3 | −3.8 | −1.2 | −5.0 |
| M16358 | Mup4 | −4.6 | −1.3 | −6.0 |
| M16360 | Mup5 | −3.6 | −1.2 | −4.7 |

Italicized fold-change identifies statistically significant intervention group;
*interaction between DF and CR
^{†}Fold change for DF, CR and DF and CR together are calculated as described in the Examples section.

TABLE 3
Complete list of the effects of DF, CR and DF and CR together on hepatic gene expression

| | | | | | |
|---|---|---|---|---|---|
| GenBank | Gene Name | Gene Symbol | DF | CR | DFCR |

Nucleotide Metabolism

| | | | | | |
|---|---|---|---|---|---|
| K01515* | hypoxanthine guanine phosphoribosyl | Hprt | 1.3^{†} | 1.2 | 1.6 |
| | transferase | | | | |
| M74495 | adenylosuccinate synthetase, muscle | Adss | 1.6 | 1.1 | 1.7 |
| AW061337 | adenylate kinase 4 | Ak4 | 1.4 | 1.2 | 1.8 |
| U49385 | cytidine 5′-triphosphate synthase 2 | Ctps2 | 1.2 | 1.1 | 1.4 |
| AW122933 | ectonucleotide | Enpp2 | 2.6 | 1.2 | 3.6 |
| | pyrophosphatase/phosphodiesterase 2 | | | | |

Energy Metabolism
Glycolysis

| | | | | | |
|---|---|---|---|---|---|
| L41631 | glucokinase | Gck | −1.3 | −1.1 | −1.5 |
| D63764 | pyruvate kinase liver and red blood cell | Pklr | −1.5 | −1.2 | −1.9 |

Gluconeogenesis Related

| | | | | | |
|---|---|---|---|---|---|
| AF009605 | phosphoenolpyruvate carboxykinase 1, | Pck1 | 1.3 | 1.2 | 1.6 |
| | cytosolic | | | | |
| AB027012 | galactokinase 1 | Galk1 | 1.4 | 1.1 | 1.7 |
| AI851321 | UDP-glucose pyrophosphorylase 2 | Ugp2 | 1.4 | 1.1 | 1.5 |
| U09114 | glutamate-ammonia ligase (glutamine | Glu1 | −1.4 | 1.0 | −1.4 |
| | synthase) | | | | |
| M14220 | glucose phosphate isomerase 1 | Gpi1 | 1.1 | 1.2 | 1.4 |
| AF080469 | glucose-6-phosphatase, transport protein 1 | G6pt1 | 1.0 | 1.3 | 1.3 |

Protein and Amino Acid Turnover

| | | | | | |
|---|---|---|---|---|---|
| AI194855 | tryptophan 2,3-dioxygenase | Tdo2 | 1.3 | 1.2 | 1.5 |
| D50586 | tissue factor pathway inhibitor 2 | Tfpi2 | −1.6 | −1.3 | −2.2 |
| U59807 | cystatin B | Cstb | 1.5 | 1.1 | 1.6 |
| M65736 | murinoglobulin 1 | Mug1 | 1.3 | 1.1 | 1.4 |
| AW047653 | ubiquitin specific protease 18 | Usp18 | 1.5 | 1.2 | 1.9 |
| AJ242663 | cathepsin Z | Ctsz | −1.1 | 1.2 | 1.2 |

TCA Cycle and Respiratory Chain

| | | | | | |
|---|---|---|---|---|---|
| AF080580 | demethyl-Q 7 | Coq7 | 1.3 | 1.3 | 1.6 |
| U51167 | isocitrate dehydrogenase 2 (NADP+), | Idh2 | 1.3 | 1.2 | 1.5 |
| | mitochondrial | | | | |
| AI854285 | influenza virus NS1A binding protein | Ivns1abp | 1.6 | 1.3 | 2.1 |
| AI851220 | cytochrome c oxidase subunit VIIb | Cox7b | 1.2 | 1.1 | 1.4 |
| AW124813 | dihydrolipoamide S-acetyltransferase (E2 | Dlat | 1.0 | 1.2 | 1.2 |
| | component of pyruvate dehydrogenase | | | | |
| | complex) | | | | |
| D50430 | glycerol phosphate dehydrogenase 2, | Gpd2 | −1.1 | −1.4 | −1.5 |
| | mitochondrial | | | | |

Lipid Uptake

| | | | | | |
|---|---|---|---|---|---|
| Z22216 | apolipoprotein C-II | Apoc2 | 1.4 | 1.3 | 1.8 |
| X58426 | lipase, hepatic | Lipc | −1.6 | −1.2 | −2.1 |
| AI846600 | monoglyceride lipase | Mgl1 | −1.4 | −1.1 | −1.6 |
| U37799 | scavenger receptor class B, member 1 | Scarb1 | 1.4 | 1.1 | 1.6 |
| Z31689 | lysosomal acid lipase 1 | Lip1 | −1.2 | −1.4 | −1.7 |

Lipid Transport

| | | | | | |
|---|---|---|---|---|---|
| M64248 | apolipoprotein A-IV | Apoa4 | −4.3 | 1.3 | −3.6 |
| U28960 | phospholipid transfer protein | Pltp | −1.7 | 1.0 | −1.7 |
| AF015790 | phospholipid scramblase 2 | Plscr2 | −1.4 | 1.0 | −1.4 |
| Y14004 | cytosolic acyl-CoA thioesterase 1 | Cte1 | −1.4 | 1.3 | −1.1 |
| M65034 | fatty acid binding protein 2, intestinal | Fabp2 | −1.8 | −1.1 | −2.0 |
| X61431 | diazepam binding inhibitor | Dbi | −1.2 | −1.1 | −1.4 |
| AF003348 | Niemann Pick type C1 | Npc1 | 1.1 | 1.3 | 1.5 |

Fatty Acid Synthesis (Lipogenesis)

| | | | | | |
|---|---|---|---|---|---|
| AI839004 | ELOVL family member 6, elongation of | Elov16 | −2.5 | −2.3 | −4.8 |
| | long chain fatty acids (yeast) | | | | |
| AW121639 | ATP citrate lyase | Acly | −1.7 | −1.2 | −2.0 |
| J02652 | malic enzyme, supernatant | Mod1 | −2.0 | −1.1 | −2.3 |
| X13135 | fatty acid synthase | Fasn | −2.3 | −1.6 | −3.6 |
| X95279 | thyroid hormone responsive SPOT14 | Thrsp | −2.1 | −1.5 | −3.2 |
| | homolog (Rattus) | | | | |

Cholesterol Synthesis

| | | | | | |
|---|---|---|---|---|---|
| X97755 | phenylalkylamine Ca2+ antagonist | Ebp | −1.4 | −1.2 | −1.6 |
| | (emopamil) binding protein | | | | |
| AW045533 | farnesyl diphosphate synthetase | Fdps | −1.9 | −1.2 | −2.3 |
| D42048 | squalene epoxidase | Sqle | −1.6 | −1.2 | −2.0 |
| AW122260 | cytochrome P450, 51 | Cyp51 | −1.6 | 1.0 | −1.6 |
| AW106745 | NAD(P) dependent steroid | Nsdhl | −1.7 | −1.2 | −2.2 |
| | dehydrogenase-like | | | | |
| AF057368 | 7-dehydrocholesterol reductase | Dhcr7 | −1.6 | 1.0 | −1.7 |

Beta-oxidation

| | | | | | |
|---|---|---|---|---|---|
| U96116 | hydroxyacyl-Coenzyme A | Hadh2 | 1.2 | 1.2 | 1.5 |
| | dehydrogenase type II | | | | |
| Y11638 | cytochrome P450, 4a14 | Cyp4a14* | 1.2 | 2.5 | 11.1 |
| U89906 | alpha-methylacyl-CoA racemase | Amacr | 1.2 | 1.2 | 1.4 |
| U07159 | acetyl-Coenzyme A dehydrogenase, | Acadm | 1.2 | 1.1 | 1.4 |
| | medium chain | | | | |
| AB018421 | cytochrome P450, 4a10 | Cyp4a10 | 1.1 | 1.8 | 2.4 |
| AI840013 | peroxisomal delta3, delta2-enoyl- | Peci | 1.1 | 1.3 | 1.5 |
| | Coenzyme A isomerase | | | | |
| AW122615 | hydroxyacyl-Coenzyme A | Hadhb | −1.1 | 1.3 | 1.2 |
| | dehydrogenase/3-ketoacyl-Coenzyme A | | | | |
| | thiolase/enoyl-Coenzyme A hydratase | | | | |
| | (trifunctional protein), beta subunit | | | | |
| AF017175 | carnitine palmitoyltransferase 1, liver | Cpt1a | 1.1 | 1.3 | 1.4 |

Others

| | | | | | |
|---|---|---|---|---|---|
| M32032 | selenium binding protein 1 | Selenbp1 | 1.3 | 1.2 | 1.5 |
| AJ011080 | afamin | Afm | −1.3 | 1.0 | −1.4 |
| AA734444 | biotinidase | Btd | −1.3 | −1.2 | −1.6 |
| AB030505 | retinol dehydrogenase 11 | Rdh11 | −1.6 | −1.1 | −1.8 |
| AF090686 | transcobalamin 2 | Tcn2 | −1.3 | −1.1 | −1.4 |
| Y15003 | sialyltransferase 9 (CMP- | Siat9 | −1.7 | 1.3 | −1.4 |
| | NeuAc: lactosylceramide alpha-2,3- | | | | |
| | sialyltransferase) | | | | |
| U05837 | hexosaminidase A | Hexa | 1.4 | 1.0 | 1.4 |
| M12330 | ornithine decarboxylase, structural | Odc | −1.2 | −1.1 | −1.4 |
| X51971 | carbonic anhydrase 5a, mitochondrial | Car5a | −1.2 | −1.2 | −1.5 |
| AB005450 | carbonic anhydrase 14 | Car14 | −1.2 | −1.2 | −1.4 |
| AI839138 | thioredoxin interacting protein | Txnip | −1.1 | 1.5 | 1.4 |
| U86108 | nicotinamide N-methyltransferase | Nnmt | −1.2 | −1.5 | −1.8 |
| U32197 | folylpolyglutamyl synthetase | Fpgs | 1.0 | −1.2 | −1.2 |

Xenobiotic and Oxidant Metabolism
Phase I

| | | | | | |
|---|---|---|---|---|---|
| M27168 | cytochrome P450, 2d9 | Cyp2d9 | 1.5 | 1.1 | 1.6 |
| M77497 | cytochrome P450, 2f2 | Cyp2f2 | −1.9 | −1.4 | −2.8 |
| D26137 | cytochrome P450, 3a16 | Cyp3a16 | 1.3 | 1.2 | 1.6 |
| U90535 | flavin containing monooxygenase 5 | Fmo5 | 1.3 | 1.3 | 1.7 |
| Y11995 | cytochrome P450, 3a25 | Cyp3a25 | 1.6 | 1.2 | 1.9 |
| U36993 | cytochrome P450, 7b1 | Cyp7b1 | −1.4 | 1.0 | −1.4 |
| AI839690 | cytochrome b5 reductase 1 (B5R.1) | Cyb5r1- | 1.1 | 1.3 | 1.4 |
| | | pending | | | |
| AI114881 | cytochrome P450, 2j5 | Cyp2j5 | −1.2 | −1.7 | −2.1 |

Phase II

| | | | | | |
|---|---|---|---|---|---|
| U89491 | epoxide hydrolase 1, microsomal | Ephx1 | 1.2 | 1.2 | 1.5 |
| Z37107 | epoxide hydrolase 2, cytoplasmic | Ephx2 | 1.3 | 1.2 | 1.6 |
| J03952 | glutathione S-transferase, mu 1 | Gstm1 | 1.2 | 1.1 | 1.4 |
| J03953 | glutathione S-transferase, mu 3 | Gstm3 | 1.5 | 1.3 | 2.0 |
| X53451 | glutathione S-transferase, pi 2 | Gstp2 | 1.6 | 1.4 | 2.6 |
| J03958 | glutathione S-transferase, alpha 2 (Yc2) | Gsta2 | 2.3 | 1.1 | 2.8 |
| L06047 | glutathione S-transferase, alpha 4 | Gsta4 | 1.5 | 1.1 | 1.8 |
| X98056 | glutathione S-transferase, theta 2 | Gstt2 | 1.1 | 1.3 | 1.5 |

Anti-oxidant

| | | | | | |
|---|---|---|---|---|---|
| U85414 | glutamate-cysteine ligase, catalytic | Gclc | 1.2 | 1.2 | 1.6 |
| | subunit | | | | |
| D87896 | glutathione peroxidase 4 | Gpx4 | 1.2 | 1.1 | 1.4 |
| AF032714 | thioredoxin peroxidase, pseudogene 1 | Tdpx-ps1 | 1.2 | 1.1 | 1.4 |
| AI851983 | glutathione reductase 1 | Gsr | 1.1 | 1.2 | 1.4 |

Others

| | | | | | |
|---|---|---|---|---|---|
| AI852001 | glyoxalase I | Glo1* | 1.0 | −1.2 | −1.3 |
| L11333 | carboxyesterase | Es31 | −5.3 | −1.3 | −6.4 |
| V00835 | metallothionein 1 | Mt1 | 1.7 | −1.4 | 1.2 |
| M88694 | thioether S-methyltransferase | Temt | 1.5 | 1.1 | 1.7 |
| AF037044 | thiopurine methyltransferase | Tpmt | 1.3 | 1.1 | 1.5 |
| AJ245750 | alcohol dehydrogenase 4 (class II), pi | Adh4 | 1.2 | 1.2 | 1.5 |
| | polypeptide | | | | |

Signal Transduction
Growth Related

| | | | | | |
|---|---|---|---|---|---|
| M22957 | prolactin receptor | Prlr | −3.0 | −1.3 | −4.5 |
| X04480 | insulin-like growth factor 1 | Igf1 | −7.2 | −1.1 | −9.7 |
| X81580 | insulin-like growth factor binding protein 2 | Igfbp2 | 1.6 | 1.7 | 3.0 |
| U57524 | nuclear factor of kappa light chain gene | Nfkbia | 1.2 | 1.2 | 1.5 |
| | enhancer in B-cells inhibitor, alpha | | | | |
| D17444 | leukemia inhibitory factor receptor | Lifr* | −3.5 | −1.7 | −5.0 |
| AI853531 | mitogen-inducible gene 6 protein | Mig6* | −1.6 | 1.2 | −1.3 |
| | homolog (Mig-6). | | | | |
| U88327 | suppressor of cytokine signaling 2 | Socs2 | −2.7 | 1.0 | −2.8 |
| X81579 | insulin-like growth factor binding protein 1 | Igfbp1 | 7.2 | 1.5 | 11.6 |
| D50086 | neuropilin | Nrp | −1.4 | −1.1 | −1.6 |
| X61597 | serine (or cysteine) proteinase inhibitor, | Serpina3c | −1.6 | 1.1 | −1.5 |
| | clade A, member 3C | | | | |
| L19932 | transforming growth factor, beta induced | Tgfbi | 1.5 | 1.1 | 1.6 |
| U92437 | phosphatase and tensin homolog | Pten | 1.1 | −1.7 | −1.6 |
| U50413 | phosphatidylinositol 3-kinase, regulatory | Pik3r1 | −1.1 | −1.6 | −1.7 |
| | subunit, polypeptide 1 (p85 alpha) | | | | |
| U39066 | mitogen activated protein kinase kinase 6 | Map2k6 | 1.1 | 1.3 | 1.5 |
| AW124627 | protein kinase C, nu | Prkcn | 1.1 | 1.2 | 1.4 |

Cytokine and Others

| | | | | | |
|---|---|---|---|---|---|
| AB017616 | Ras-related GTP binding C | Rragc | 1.1 | 1.2 | 1.4 |
| AJ245569 | Rab6 interacting protein 1 | Rab6ip1 | 1.2 | 1.1 | 1.4 |
| Y12738 | adrenergic receptor, alpha 1b | Adra1b | −1.2 | −1.2 | −1.5 |
| L21221 | proprotein convertase subtilisin/kexin | Pcsk4 | 1.2 | 1.3 | 1.7 |
| | type 4 | | | | |
| Y09517 | hydroxysteroid (17-beta) dehydrogenase 2 | Hsd17b2 | −2.1 | −1.4 | −3.0 |
| AA822174 | retinal short-chain | Retsdr2- | 1.4 | 1.3 | 2.0 |
| | dehydrogenase/reductase 2 | pending | | | |
| M77015 | hydroxysteroid dehydrogenase-3, | Hsd3b3 | 1.3 | 1.1 | 1.4 |
| | delta<5>-3-beta | | | | |
| AF031170 | hydroxysteroid dehydrogenase-6, | Hsd3b6 | 1.4 | 1.2 | 1.6 |
| | delta<5>-3-beta | | | | |
| M93422 | adenylate cyclase 6 | Adcy6 | 1.3 | 1.1 | 1.6 |
| Z50190 | adenylate cyclase 9 | Adcy9 | 1.3 | 1.1 | 1.4 |
| AF047727 | cytochrome P450, 2c40 | Cyp2c40 | −1.9 | 1.0 | −1.9 |
| AI047331 | cytochrome P450, family 2, subfamily c, | Cyp2c70 | −1.2 | −1.2 | −1.5 |
| | polypeptide 70 | | | | |
| AI845798 | phospholipase A2, group XII | Pla2g12 | 1.3 | 1.1 | 1.6 |
| AW125649 | guanine nucleotide binding protein, alpha | Gna12 | 1.3 | 1.1 | 1.5 |
| | 12 | | | | |
| AA608387 | interleukin 13 receptor, alpha 1 | Il13ra1 | −1.3 | −1.1 | −1.4 |
| AI272518 | Rab geranylgeranyl transferase, a subunit | Rabggta | −1.3 | 1.0 | −1.3 |
| U84411 | protein tyrosine phosphatase 4a1 | Ptp4a1 | 1.3 | 1.0 | 1.4 |
| AF004927 | opioid receptor, sigma 1 | Oprs1 | −1.3 | −1.1 | −1.5 |
| AV349152 | regulator of G-protein signaling 16 | Rgs16 | 1.1 | 2.2 | 3.1 |
| M20658 | interleukin 1 receptor, type I | Il1r1 | 1.1 | 1.4 | 1.7 |
| L09737 | GTP cyclohydrolase 1 | Gch | −1.1 | 1.3 | 1.2 |
| Y17860 | ganglioside-induced differentiation- | Gdap10 | 1.2 | −1.6 | −1.4 |
| | associated-protein 10 | | | | |

Transcription
General Transcription

| | | | | | |
|---|---|---|---|---|---|
| AI132239 | transcription elongation factor A (SII), 3 | Tcea3 | 1.4 | 1.1 | 1.5 |
| X60136 | trans-acting transcription factor 1 | Sp1 | 1.1 | −1.4 | −1.4 |
| Z47088 | S-phase kinase-associated protein 1A | Skp1a | 1.1 | −1.5 | −1.3 |

Histone Modulation

| | | | | | |
|---|---|---|---|---|---|
| AI844939 | CREBBP/EP300 inhibitory protein 1 | Cri1 | 1.3 | 1.2 | 1.7 |
| AI837110 | heterogeneous nuclear ribonucleoproteins | Hrmt1l2 | 1.3 | 1.0 | 1.4 |
| | methyltransferase-like 2 (S. cerevisiae) | | | | |
| U73478 | acidic (leucine-rich) nuclear | Anp32a | −1.1 | −2.2 | −2.5 |
| | phosphoprotein 32 family, member A | | | | |
| AW047728 | p300/CBP-associated factor | Pcaf | 1.0 | 1.3 | 1.4 |
| AA790056 | cysteine and histidine rich 1 (p300/CBP) | Cyhr1 | 1.0 | 1.3 | 1.4 |
| AF053062 | nuclear receptor interacting protein 1 | Nrip1 | 1.1 | −1.5 | −1.3 |

Transcriptional Repressor

| | | | | | |
|---|---|---|---|---|---|
| AW048812 | hairy and enhancer of split 6, | Hes6 | −1.8 | −1.3 | −2.3 |
| | (Drosophila) | | | | |
| AW047223 | O-linked N-acetylglucosamine (GlcNAc) | Ogt | 1.3 | 1.2 | 1.7 |
| | transferase (UDP-N- | | | | |
| | acetylglucosamine:polypeptide-N- | | | | |
| | acetylglucosaminyl transferase) | | | | |
| AI852535 | SCAN-KRAB-zinc finger gene 1 | Skz1-pending | 1.2 | 1.2 | 1.4 |
| L20450 | zinc finger protein 97 | Zfp97 | 1.4 | 1.2 | 1.8 |
| AW061318 | CUG triplet repeat, RNA binding protein 2 | Cugbp2 | 1.2 | 1.1 | 1.4 |
| AF091096 | RPB5-mediating protein | Rmp-pending | 1.3 | 1.0 | 1.4 |
| AW048233 | Est2 repressor factor | Erf | 1.3 | 1.0 | 1.4 |
| X89749 | TG interacting factor | Tgif | 1.4 | 1.0 | 1.4 |
| U88539 | suppressor of Ty 5 homolog (S. cerevisiae) | Supt5h | 1.3 | 1.1 | 1.4 |

Others

| | | | | | |
|---|---|---|---|---|---|
| L10409 | forkhead box A2 | Foxa2 | −1.2 | −1.2 | −1.5 |
| X74938 | forkhead box A3 | Foxa3 | 1.2 | 1.3 | 1.6 |
| AF084524 | cellular repressor of E1A-stimulated | Creg | 1.2 | 1.2 | 1.5 |
| | genes | | | | |
| U49507 | liver-specific bHLH-Zip transcription | Lisch7-pending | −1.4 | −1.1 | −1.6 |
| | factor | | | | |
| U73029 | interferon regulatory factor 6 | Irf6 | −2.5 | −1.1 | −2.8 |
| X14678 | zinc finger protein 36 | Zfp36 | 1.3 | −1.1 | 1.2 |
| AI987985 | zinc finger protein 288 | Zfp288 | −1.1 | −1.3 | −1.5 |
| L04961 | inactive X specific transcripts | Xist | 1.1 | −1.7 | −1.5 |

RNA Metabolism (RNA Splicing and Translation)

| | | | | | |
|---|---|---|---|---|---|
| AI846123 | G-rich RNA sequence binding factor 1 | Grsf1 | 1.2 | 1.2 | 1.5 |
| AF093140 | nuclear RNA export factor 1 homolog (S. cervisiae) | Nxf1 | 1.2 | 1.2 | 1.5 |
| X75895 | ribosomal protein L36 | Rpl36 | 1.1 | 1.2 | 1.4 |
| AW047116 | ribosomal protein L37 | Rpl37 | 1.1 | 1.2 | 1.4 |
| AB016424 | RNA binding motif protein 3 | Rbm3 | 2.0 | 1.6 | 3.5 |
| AI852608 | RNA cyclase homolog | Rnac-pending | 1.2 | 1.2 | 1.5 |
| AI838709 | spermatid perinuclear RNA binding | Spnr | 1.3 | 1.2 | 1.5 |
| | protein | | | | |
| AF026481 | eukaryotic translation initiation factor 1A | Eif1a | 1.5 | 1.1 | 1.6 |
| D78135 | cold inducible RNA binding protein | Cirbp | 1.2 | 1.1 | 1.4 |
| AI844131 | heterogeneous nuclear ribonucleoprotein | Hnrpa2b1 | 1.3 | 1.1 | 1.5 |
| | A2/B1 | | | | |
| AI840339 | ribonuclease, RNase A family 4 | Rnase4 | 1.3 | 1.0 | 1.4 |
| X97982 | poly(rC) binding protein 2 | Pcbp2 | 1.2 | 1.1 | 1.4 |
| AI849620 | threonyl-tRNA synthetase | Tars | −1.6 | 1.0 | −1.6 |
| M38381 | CDC-like kinase | Clk | 1.1 | 1.2 | 1.4 |
| AI844532 | splicing factor 3b, subunit 1, 155 kDa | Sf3b1 | 1.2 | −1.9 | −1.6 |
| AF095257 | heterogeneous nuclear ribonucleoprotein C | Hnrpc | 1.1 | −1.3 | −1.2 |
| AI875598 | mitochondrial translational initiation | Mtif2 | 1.0 | −1.3 | −1.3 |
| | factor | | | | |

Transport and Trafficking
Membrane Transport

| | | | | | |
|---|---|---|---|---|---|
| AI173996 | ATP-binding cassette, sub-family C | Abcc2 | 1.4 | 1.2 | 1.7 |
| | (CFTR/MRP), member 2 | | | | |
| AA833514 | ATP-binding cassette, sub-family C | Abcc3 | 1.3 | 1.1 | 1.4 |
| | (CFTR/MRP), member 3 | | | | |
| AF103875 | ATP-binding cassette, sub-family G | Abcg2 | 1.8 | 1.3 | 2.3 |
| | (WHITE), member 2 | | | | |
| AA655369 | translocase of inner mitochondrial | Timm8a | 1.1 | 1.3 | 1.4 |
| | membrane 8 homolog a (yeast) | | | | |
| AI843085 | importin 7 | Ipo7 | 1.1 | 1.2 | 1.4 |

Intracellular Transport

| | | | | | |
|---|---|---|---|---|---|
| L03290 | solute carrier family 7 (cationic amino | Slc7a2 | 1.3 | 1.1 | 1.5 |
| | acid transporter, y+ system), member 2 | | | | |
| U95132 | solute carrier family 10 (sodium/bile acid | Slc10a1 | −1.4 | 1.1 | −1.2 |
| | cotransporter family), member 1 | | | | |
| AF058054 | solute carrier family 16 (monocarboxylic | Slc16a7 | 1.5 | 1.1 | 1.7 |
| | acid transporters), member 7 | | | | |
| U38652 | solute carrier family 22 (organic cation | Slc22a1 | −1.6 | −1.2 | −2.0 |
| | transporter), member 1 | | | | |
| AI844736 | solute carrier family 23 (nucleobase | Slc23a2 | 1.4 | 1.1 | 1.6 |
| | transporters), member 2 | | | | |
| AI838274 | solute carrier family 29 (nucleoside | Slc29a1 | −1.3 | 1.0 | −1.4 |
| | transporters), member 1 | | | | |
| AW124985 | striatin, calmodulin binding protein 3 | Strn3 | 1.3 | 1.1 | 1.5 |
| U34259 | lysosomal-associated protein | Laptm4a | 1.1 | −1.3 | −1.2 |
| | transmembrane 4A | | | | |
| AF020195 | solute carrier family 4 (anion exchanger), | Slc4a4 | 1.2 | −1.5 | −1.3 |
| | member 4 | | | | |
| AW048729 | solute carrier family 5 (sodium- | Slc5a6 | 1.1 | 1.3 | 1.4 |
| | dependent vitamin transporter), member 6 | | | | |
| M73696 | solute carrier family 20, member 1 | Slc20a1 | −1.2 | 1.5 | 1.4 |

Cell Proliferation (Cell Cycle and DNA Replication)

| | | | | | |
|---|---|---|---|---|---|
| X59846 | growth arrest specific 6 | Gas6* | −1.5 | −1.4 | −2.0 |
| AI849928 | cyclin D1 | Ccnd1 | −1.6 | 1.2 | −1.3 |
| AW060791 | polymerase (DNA-directed), epsilon 4 | Pole4 | −1.3 | −1.1 | −1.5 |
| | (p12 subunit) | | | | |
| AL021127 | centrin 2 | Cetn2 | −1.7 | −1.1 | −2.1 |
| X15986 | lectin, galactose binding, soluble 1 | Lgals1 | 1.6 | 1.0 | 1.6 |
| AA913994 | serine hydroxymethyl transferase 1 | Shmt1 | −1.4 | −1.2 | −1.7 |
| | (soluble) | | | | |
| AW120896 | cysteine sulfinic acid decarboxylase | Csad | −1.6 | 1.4 | −1.2 |
| AF064088 | TGFB inducible early growth response 1 | Tieg1 | −1.3 | 1.4 | 1.1 |
| X95280 | G0/G1 switch gene 2 | G0s2 | −1.1 | −1.9 | −2.0 |
| AI840051 | cullin 3 | Cul3 | 1.1 | 1.2 | 1.4 |

Apoptosis

| | | | | | |
|---|---|---|---|---|---|
| Y13087 | caspase 6 | Casp6 | 1.2 | 1.2 | 1.4 |
| U57325 | presenilin 2 | Psen2 | −1.3 | −1.3 | −1.6 |
| M21828 | growth arrest specific 2 | Gas2 | 1.7 | 1.0 | 1.8 |
| U32170 | regucalcin | Rgn | −1.2 | −1.1 | −1.4 |

Chaperone (Protein Folding)

| | | | | | |
|---|---|---|---|---|---|
| AI846938 | homocysteine-inducible, endoplasmic | Herpud1 | 1.2 | 1.3 | 1.6 |
| | reticulum stress-inducible, ubiquitin-like | | | | |
| | domain member 1 | | | | |
| AF055664 | DnaJ (Hsp40) homolog, subfamily A, | Dnaja1 | −1.2 | −1.2 | −1.5 |
| | member 1 | | | | |
| AA615831 | heat shock protein 4 | Hspa4 | −1.4 | −1.2 | −1.7 |
| L40406 | heat shock protein 105 | Hsp105 | −1.6 | −1.1 | −1.7 |
| J04633 | heat shock protein 1, alpha | Hspca | −1.5 | −1.1 | −1.7 |
| AW122022 | peptidylprolyl isomerase D (cyclophilin | Ppid | −1.2 | −1.2 | −1.4 |
| | D) | | | | |
| AI842377 | protein disulfide isomerase-related | P5-pending | −1.4 | 1.0 | −1.4 |
| | protein | | | | |
| AV373612 | Bcl2-associated athanogene 3 | Bag3 | −1.3 | 1.0 | −1.4 |
| D17666 | heat shock protein, A | Hspa9a | 1.1 | 1.3 | 1.4 |
| AF109906 | heat shock protein 1B | Hspa1b | 1.1 | 1.5 | 1.8 |
| AA879709 | signal sequence receptor, alpha | Ssr1 | −1.1 | −1.3 | −1.5 |

Cell Adhesion and Structure Protein

| | | | | | |
|---|---|---|---|---|---|
| AI195392 | actinin, alpha 1 | Actn1 | 1.2 | 1.2 | 1.5 |
| U38196 | membrane protein, palmitoylated | Mpp1 | 1.3 | 1.2 | 1.6 |
| Z22532 | syndecan 1 | Sdc1 | −1.2 | −1.2 | −1.5 |
| AI152659 | desmoglein 2 | Dsg2 | −1.2 | −1.1 | −1.4 |
| L25274 | activated leukocyte cell adhesion | Alcam | 1.5 | −1.3 | 1.2 |
| | molecule | | | | |
| X15202 | integrin beta 1 (fibronectin receptor beta) | Itgb1 | 1.2 | 1.1 | 1.4 |
| AI462105 | vinculin | Vc1 | 1.5 | 1.1 | 1.7 |
| M21495 | actin, gamma, cytoplasmic | Actg | 1.4 | −1.2 | 1.2 |
| AF053367 | PDZ and LIM domain 1 (elfin) | Pdlim1 | −1.2 | −1.1 | −1.4 |
| AW260404 | PDZ domain containing 1 | Pdzk1 | −1.4 | 1.0 | −1.4 |
| X61172 | mannosidase 2, alpha 1 | Man2a1 | 1.3 | 1.1 | 1.4 |
| AW123026 | glucosamine-phosphate N- | Gnpnat1 | 1.4 | 1.1 | 1.5 |
| | acetyltransferase 1 | | | | |
| AI851740 | actin related protein 2/3 complex, subunit 3 | Arpc3 | 1.1 | −1.4 | −1.2 |

Matrix Protein

| | | | | | |
|---|---|---|---|---|---|
| M15832 | procollagen, type IV, alpha 1 | Col4a1 | 1.3 | 1.1 | 1.4 |
| X70391 | inter-alpha trypsin inhibitor, heavy chain 1 | Itih1 | −1.4 | −1.1 | −1.5 |

Ion Channel and Transport

| | | | | | |
|---|---|---|---|---|---|
| M81445 | gap junction membrane channel protein | Gjb2 | −1.5 | −1.4 | −2.2 |
| | beta 2 | | | | |
| AI849587 | protein distantly related to to the gamma | Pr1 | −1.3 | −1.2 | −1.6 |
| | subunit family | | | | |
| AF018952 | aquaporin 8 | Aqp8 | −2.0 | −1.3 | −2.6 |
| AF089751 | purinergic receptor P2X, ligand-gated ion | P2rx4 | 1.3 | 1.1 | 1.5 |
| | channel 4 | | | | |
| AW123952 | ATPase, Na+/K+ transporting, alpha 1 | Atp1a1 | 1.1 | −1.4 | −1.4 |
| | polypeptide | | | | |

Immune System

| | | | | | |
|---|---|---|---|---|---|
| U09010 | mannose binding lectin, liver (A) | Mbl1 | −1.4 | −1.2 | −1.7 |
| U09016 | mannose binding lectin, serum (C) | Mbl2 | −1.5 | −1.1 | −1.7 |
| X17069 | FK506 binding protein 4 | Fkbp4 | −1.3 | −1.2 | −1.7 |
| U16959 | FK506 binding protein 5 | Fkbp5 | −1.9 | 1.4 | −1.4 |
| J04596 | chemokine (C—X—C motif) ligand 1 | Cxcl1 | 3.5 | −1.1 | 3.4 |
| Z16410 | B-cell translocation gene 1, anti- | Btg1 | 1.5 | 1.0 | 1.4 |
| | proliferative | | | | |
| U41465 | B-cell leukemia/lymphoma 6 | Bcl6 | 2.0 | 1.2 | 2.5 |
| M57891 | complement component 2 (within H—2S) | C2 | 1.3 | 1.0 | 1.4 |
| AI118358 | histidine-rich glycoprotein | Hrg | 1.3 | 1.0 | 1.4 |
| X56135 | prothymosin alpha | Ptma | 1.4 | 1.0 | 1.4 |
| AB007813 | ficolin A | Fcna | −1.4 | −1.1 | −1.5 |
| D16492 | mannan-binding lectin serine protease 1 | Masp1 | −1.3 | 1.1 | −1.2 |
| D88577 | C-type (calcium dependent, carbohydrate | Clecsf13 | −1.2 | −1.1 | −1.4 |
| | recognition domain) lectin, superfamily | | | | |
| | member 13 | | | | |
| AA986114 | T-cell immunoglobulin and mucin | Timd2 | −1.5 | 1.0 | −1.6 |
| | domain containing 2 | | | | |
| AA268823 | CD59b antigen | Cd59b | −1.3 | −1.1 | −1.5 |

Pheromone

| | | | | | |
|---|---|---|---|---|---|
| M17818 | major urinary protein 1 | Mup1 | −4.6 | −1.2 | −7.4 |
| M16357 | major urinary protein 3 | Mup3 | −3.8 | −1.2 | −5.0 |
| M16358 | major urinary protein 4 | Mup4 | −4.6 | −1.3 | −6.0 |
| M16360 | major urinary protein 5 | Mup5 | −3.6 | −1.2 | −4.7 |

Neurotransmitter

| | | | | | |
|---|---|---|---|---|---|
| AW123904 | gamma-aminobutyric acid (GABA(A)) | Gabarapl1 | 1.3 | 1.1 | 1.5 |
| | receptor-associated protein-like 1 | | | | |
| AW212131 | synaptonemal complex protein 3 | Sycp3 | 2.0 | 1.1 | 2.4 |
| AF093259 | homer homolog 2 (Drosophila) | Homer2 | −1.6 | 1.0 | −1.6 |
| AF071068 | dopa decarboxylase | Ddc | −1.1 | −1.5 | −1.6 |

Miscellaneous

| | | | | | |
|---|---|---|---|---|---|
| AW123662 | secretory carrier membrane protein 1 | Scamp1 | 1.2 | 1.3 | 1.6 |
| U73039 | neighbor of Brca1 gene 1 | Nbr1 | 1.1 | 1.2 | 1.4 |
| X73523 | sialyltransferase 4A (beta-galactosidase | Siat4a | −1.2 | −1.3 | −1.6 |
| | alpha-2,3-sialytransferase) | | | | |
| AF039663 | prominin-like 1 | Proml1 | −1.7 | −1.5 | −2.4 |
| AI840971 | brain protein 17 | Brp17 | −1.1 | −1.3 | −1.4 |
| AW125626 | calponin 3, acidic | Cnn3 | 1.3 | 1.2 | 1.5 |
| AI840501 | camello-like 1 | Cml1 | −1.4 | −1.2 | −1.7 |
| Z54179 | gene trap locus 3 | Gtl3 | 1.2 | 1.2 | 1.5 |
| Z50159 | suppressor of initiator codon mutations, | Suil-rs1 | 1.2 | 1.3 | 1.6 |
| | related sequence 1 (S. cerevisiae) | | | | |
| U44088 | pleckstrin homology-like domain, family | Phlda1 | −2.0 | −1.8 | −3.1 |
| | A, member 1 | | | | |
| AI853773 | F-box only protein 21 | Fbxo21 | 1.9 | 1.6 | 3.7 |
| AW047445 | transmembrane 7 superfamily member 2 | Tm7sf2 | −1.3 | −1.3 | −1.6 |
| AI843802 | lipin 2 | Lpin2 | 1.2 | 1.4 | 1.7 |
| AJ009840 | cathepsin E | Ctse | −1.4 | 1.3 | −1.0 |
| D64162 | retinoic acid early transcript gamma | Raet1c | 1.5 | 1.0 | 1.6 |
| M96827 | haptoglobin | Hp | 1.3 | 1.0 | 1.4 |
| AF087687 | S100 calcium binding protein A1 | S100a1 | 1.3 | 1.1 | 1.5 |
| M16465 | S100 calcium binding protein A10 | S100a10 | −1.9 | −1.2 | −2.4 |
| | (calpactin) | | | | |
| AI225445 | DNA cross-link repair 1A, PSO2 | Dclre1a | 1.8 | 1.0 | 1.8 |
| | homolog (S. cerevisiae) | | | | |
| AI507104 | gamma-glutamyl carboxylase | Ggcx | −1.2 | −1.1 | −1.4 |
| M29961 | glutamyl aminopeptidase | Enpep | −1.4 | 1.0 | −1.4 |
| AI837311 | nuclear distribution gene E-like homolog | Ndel1 | 1.4 | 1.2 | 1.6 |
| | 1 (A. nidulans) | | | | |
| M15268 | aminolevulinic acid synthase 2, erythroid | Alas2 | 1.5 | −1.1 | 1.4 |
| X73230 | arylsulfatase A | Arsa | 2.7 | 1.0 | 2.8 |
| M23552 | serum amyloid P-component | Apcs | −2.0 | 1.2 | −1.7 |
| M93275 | adipose differentiation related protein | Adfp | −1.2 | −1.1 | −1.4 |
| Z38015 | dystrophia myotonica kinase, B15 | Dm15 | 1.4 | 1.1 | 1.4 |
| AB028071 | kidney expressed gene 1 | Keg1 | −2.4 | −1.2 | −2.9 |
| AW120606 | carcinoma related gene | Flana-pending | −1.2 | −1.1 | −1.4 |
| Z31362 | neoplastic progression 3 | Npn3 | 1.6 | 1.1 | 1.8 |
| AF033186 | WD-40-repeat-containing protein with a | Wsb1-pending | −1.4 | 1.0 | −1.3 |
| | SOCS box 1 | | | | |
| AI851250 | sprouty protein with EVH-1 domain 2, | Spred2 | 1.3 | 1.0 | 1.4 |
| | related sequence | | | | |
| AI852098 | ELOVL family member 5, elongation of | Elovl5 | −1.4 | −1.2 | −1.7 |
| | long chain fatty acids (yeast) | | | | |
| AI854794 | tensin like C1 domain-containing | Tenc1 | 1.3 | 1.1 | 1.4 |
| | phosphatase | | | | |
| AW046579 | F-box only protein 3 | Fbxo3 | 1.2 | 1.1 | 1.4 |
| AW212859 | axotrophin | Axot | 1.3 | 1.0 | 1.3 |
| U43285 | selenophosphate synthetase 2 | Sps2 | 1.3 | 1.1 | 1.4 |
| U82624 | amyloid beta (A4) precursor protein | App | 1.3 | 1.1 | 1.4 |
| U61183 | yolk sac gene 2 | Ysg2 | 1.0 | −1.3 | −1.3 |
| AJ007909 | erythroid differentiation regulator | Erdr1-pending | 1.0 | 1.5 | 1.6 |
| AA597220 | regulator of chromosome condensation | Rcbtb1 | 1.0 | 1.4 | 1.5 |
| | (RCC1) and BTB (POZ) domain | | | | |
| | containing protein 1 | | | | |
| AV299153 | DEAH (Asp-Glu-Ala-His) box | Dhx36 | 1.1 | −1.4 | −1.3 |
| | polypeptide 36 | | | | |

Opposite Direction

| | | | | | |
|---|---|---|---|---|---|
| AI846934 | lipin 1 | Lpin1 | −1.7 | 1.8 | 1.1 |
| U87147 | flavin containing monooxygenase 3 | Fmo3 | −2.3 | 1.5 | −1.5 |
| Y12657 | cytochrome P450, 26, a1 | Cyp26a1 | 1.9 | −1.4 | 1.3 |
| K02236 | metallothionein 2 | Mt2 | 1.7 | −1.7 | −1.0 |
| AI842603 | YY1 transcription factor | Yy1 | −1.3 | 1.2 | −1.0 |

EST

| | | | | |
|---|---|---|---|---|
| AW047554 | RIKEN cDNA 1110001I14 gene | −1.6 | −1.2 | −2.1 |
| AW215585 | RIKEN cDNA 9130422G05 gene | −1.5 | −1.2 | −1.9 |
| AW125421 | EST | 1.1 | 1.2 | 1.4 |
| AW124049 | EST | 1.4 | 1.4 | 2.3 |
| AW122893 | RIKEN cDNA 1810015C04 gene | 1.3 | 1.3 | 1.8 |
| AW061234 | RIKEN cDNA A230075M04 gene | −1.8 | −1.5 | −2.5 |
| AW047919 | hypothetical protein C130003G01 | 1.3 | 1.2 | 1.6 |
| AW046449 | RIKEN cDNA 2600014B10 gene | 1.3 | 1.2 | 1.7 |
| AI854771 | RIKEN cDNA E230009N18 gene | 1.2 | 1.2 | 1.4 |
| AI854482 | Similar to KIAA0268 protein | 1.2 | 1.2 | 1.5 |
| AI851798 | Similar to general transcription factor Iia | 1.2 | 1.2 | 1.5 |
| AI848584 | RIKEN cDNA 1110002B05 gene | 1.3 | 1.2 | 1.7 |
| AI843959 | RIKEN cDNA 5730403B10 gene | 1.2 | 1.4 | 1.7 |
| AI842264 | RIKEN cDNA 2610311I19 gene | −1.2 | −1.1 | −1.4 |
| AI662099 | EST | −1.5 | −1.3 | −1.9 |
| AI553401 | clone MGC: 57103 IMAGE: 6491688 | 1.2 | 1.4 | 1.8 |
| AI461631 | RIKEN cDNA 1110025G12 gene | −1.1 | −1.2 | −1.4 |
| AI414025 | RIKEN cDNA 2900016D05 gene | −1.3 | −1.1 | −1.4 |
| AI047107 | RIKEN cDNA 3732413I11 gene | 1.6 | 1.3 | 2.4 |
| AA960603 | Brf2 gene, 3′ UTR | 1.3 | 1.1 | 1.6 |
| AA798246 | EST | 1.2 | 1.2 | 1.4 |
| AA670737 | RIKEN cDNA 1700013L23 gene | −1.3 | −1.1 | −1.5 |
| X90778 | EST | −1.2 | −1.1 | −1.4 |
| X04097 | EST | 1.4 | 1.1 | 1.6 |
| M80423 | EST | 1.5 | −1.2 | 1.3 |
| M17551 | EST | 1.5 | 1.2 | 1.8 |
| M10062 | EST | 1.4 | 1.2 | 1.7 |
| AW212475 | RIKEN cDNA 1300002F13 gene | −1.7 | 1.1 | −1.5 |
| AW209004 | EST | 1.3 | 1.2 | 1.5 |
| AW125508 | RIKEN cDNA 1110029F20 gene | −1.5 | −1.1 | −1.7 |
| AW125453 | RIKEN cDNA 1190002N15 gene | 1.3 | 1.1 | 1.5 |
| AW124122 | RIKEN cDNA 2010200I23 gene | −1.4 | 1.0 | −1.4 |
| AW123751 | RIKEN cDNA 2310056P07 gene | 1.2 | 1.1 | 1.4 |
| AW123249 | hypothetical protein MGC12117 | 1.3 | 1.1 | 1.4 |
| AW123061 | RIKEN clone: 9930106P14 | −1.2 | −1.1 | −1.4 |
| AW121496 | RIKEN cDNA 1810005H09 gene | −1.3 | −1.1 | −1.5 |
| AW060549 | Moderately similar to A47643 | 1.7 | 1.4 | 2.3 |
| AW060358 | RIKEN cDNA B430110G05 gene | −1.5 | 1.0 | −1.5 |
| AW048053 | EST | 1.2 | 1.1 | 1.4 |
| AV251443 | EST | 1.6 | 1.0 | 1.7 |
| AI854331 | RIKEN cDNA A030007L17 gene | −1.2 | −1.1 | −1.4 |
| AI853364 | EST | −1.3 | −1.2 | −1.5 |
| AI853226 | clone IMAGE: 4237666 | 1.3 | 1.0 | 1.4 |
| AI850090 | RIKEN cDNA 5730469M10 gene | 1.4 | 1.1 | 1.6 |
| AI848479 | EST | 1.5 | 1.1 | 1.7 |
| AI845538 | EST | −1.3 | −1.2 | −1.5 |
| AI842544 | RIKEN cDNA 2310044G17 gene | −1.6 | 1.0 | −1.6 |
| AI842065 | RIKEN clone: E330037P08 | 1.6 | 1.2 | 1.9 |
| AI841894 | EST | −1.5 | 1.2 | −1.3 |
| AI841330 | EST | −1.4 | 1.0 | −1.5 |
| AI837302 | RIKEN cDNA 1010001C05 gene | 1.2 | 1.1 | 1.4 |
| AI836143 | RIKEN cDNA 1500036F01 gene | 2.2 | −1.3 | 2.0 |
| AI787183 | RIKEN cDNA 0610011I04 gene | 1.6 | 1.0 | 1.5 |
| AI647632 | RIKEN cDNA C730048C13 gene | −1.4 | −1.1 | −1.5 |
| AI157548 | RIKEN cDNA 3110004O18 gene | 1.2 | 1.2 | 1.4 |
| AI049144 | RIKEN cDNA 1300013B24 gene | −1.6 | 1.1 | −1.5 |
| AF031380 | RIKEN cDNA 0610038L10 gene | −1.5 | −1.2 | −1.7 |
| AB031386 | RIKEN cDNA 1810009M01 gene | 1.4 | 1.0 | 1.5 |
| AA981581 | RIKEN clone: A430083K13 | 1.3 | 1.1 | 1.4 |
| AA914105 | RIKEN cDNA 2310075C12 gene | 1.4 | 1.0 | 1.4 |
| AA755234 | RIKEN cDNA 9030612M13 gene | 1.4 | 1.2 | 1.7 |
| AA710439 | RIKEN cDNA 6230421P05 gene | 1.3 | 1.1 | 1.4 |
| AA710132 | RIKEN cDNA 1100001H23 gene | −1.4 | 1.1 | −1.3 |
| AA656550 | RIKEN cDNA 1300006M19 gene | 1.0 | 1.2 | 1.3 |
| D87691 | EST | 1.0 | 1.2 | 1.3 |
| AW121568 | RIKEN cDNA 3110001N18 gene | 1.0 | 1.3 | 1.3 |
| AW047688 | RIKEN cDNA 0610039N19 gene | −1.1 | 1.8 | 2.0 |
| AW046723 | RIKEN cDNA 2400003B06 gene | −1.1 | −1.2 | −1.4 |
| AI854813 | EST | −1.1 | −1.4 | −1.6 |
| AI849075 | RIKEN cDNA 1500041O16 gene | −1.1 | −1.4 | −1.6 |
| AI846522 | RIKEN cDNA B930035K21 gene | 1.0 | 1.3 | 1.3 |
| AI840615 | RIKEN cDNA 5730472N09 gene | −1.1 | −1.2 | −1.4 |
| AI836322 | RIKEN cDNA 6720463E02 gene | 1.0 | −1.6 | −1.6 |
| AI787317 | Highly similar to apolipoprotein B-100 | 1.3 | −2.1 | −1.6 |
| AI647548 | EST | 1.1 | 1.4 | 1.6 |
| AI553024 | strong similarity to human Zinc finger | 1.1 | 2.1 | 2.3 |
| | protein 145 | | | |
| AI425990 | RIKEN cDNA C530046L02 gene | 1.0 | 1.2 | 1.3 |
| AI272489 | RIKEN cDNA E130315B21 gene | 1.0 | 1.2 | 1.2 |
| AI194254 | EST | 1.0 | 1.3 | 1.4 |
| AI173533 | EST | 1.0 | 1.3 | 1.4 |
| AI153421 | clone MGC: 46985 IMAGE: 5004588 | 1.1 | 1.4 | 1.6 |
| AI037493 | RIKEN cDNA 4432405K22 gene | 1.1 | 1.2 | 1.4 |
| AA815795 | RIKEN cDNA 1200007D18 gene | −1.1 | −1.4 | −1.5 |

Italicized: fold-change identifies statistically significant intervention group;
*interaction between DF and CR
^{†}Fold change for DF, CR and DF and CR together are calculated as described in Examples section information.

1. A method of identifying an intervention that modulates a biomarker of aging, the method comprising:
exposing a biological sample to a test intervention;
measuring the level of a gene product set forth in Table 3; and
identifying a change in the level of the gene product that correlates with a change observed in dwarfism, caloric-restriction or both caloric-restriction and dwarfism, thereby identifying an intervention that modulates a biomarker of aging.

2. The method of claim 1, wherein the gene product is set forth in Table 2.

3. The method of claim 1, wherein the gene product is modulated in dwarf mice.

4. The method of claim 1, wherein the gene product is modulated in caloric-restricted mice.

5. The method of claim 1, wherein the gene product is modulated in caloric-restricted dwarf mice.

6. The method of claim 1, wherein the biological sample is an animal.

7. The method of claim 6, wherein the animal is a mouse.

8. The method of claim 1, wherein the biological sample comprises cells isolated from a subject.

9. The method of claim 8, wherein the cells comprise liver cells.

10. The method of claim 1, wherein the gene product is a member of a signal transduction cascade.

11. The method of claim 1, wherein the gene product plays a role in apoptosis.

12. The method of claim 1, wherein the gene product is a chaperone.

13. The method of claim 1, wherein the gene product plays a role in glucose metabolism.

14. The method of claim 1, wherein the gene product plays a role in lipid metabolism.

15. The method of claim 1, wherein the gene product plays a role in oxidant and toxin defense.

16. The method of claim 1, wherein the step of measuring the level of the gene product comprises measuring the level of mRNA.

17. The method of claim 1, wherein the step of measuring the level of the gene product comprises measuring the level of protein.

18. The method of claim 1, wherein the step of measuring the level of gene product comprises measuring protein activity.

19. The method of claim 1, wherein the step of measuring the level of gene product comprises measuring protein modifications.

20. A method of identifying a biomarker of aging, the method comprising:
subjecting a dwarf mouse to a caloric-restricted diet;
comparing an expression profile of a biological sample from the dwarf mouse to the expression profile of a control-fed normal mouse and a control-fed dwarf mouse, and
identifying changes in the expression profile that occur in the caloric-restricted dwarf mouse relative to the control-fed normal and dwarf mice.

21. The method of claim 20, further comprising a step of comparing the expression profile of the caloric-restricted dwarf mouse to an expression profile from a normal mouse that is subjected to caloric restriction and identifying changes in the expression profile that occur in the caloric-restricted dwarf mouse relative to the caloric-restricted normal mouse.

22. The method of claim 20, wherein the step of comparing the expression profile from the caloric restricted mouse to that of the control-fed mice comprises measuring levels of RNA.

23. The method of claim 20, wherein the expression profile is determined using an oligonucleotide-based high density array.

24. The method of claim 20, wherein the step of comparing the expression profile from the caloric-restricted mouse to that of the expression profile of the control-fed mice comprises measuring levels of protein.

25. The method of claim 20, wherein the step of comparing the expression profile from the caloric-restricted mouse to that of the expression profile of the control-fed mice comprises measuring protein activity.

26. The method of claim 20, wherein the step of comparing the expression profile from the caloric-restricted mouse to that of the expression profile of the control-fed mice comprises measuring the levels of protein modification.

27. The method of claim 20, wherein the expression profile is obtained using liver tissue.

28. The method of claim 20, wherein the dwarf mouse is subjected to short-term caloric restriction.

29. A method of identifying an intervention that modulates a biomarker of longevity, the method comprising:
exposing a biological sample to a test intervention;
measuring the level of a gene product identified in accordance with claim 20; and
identifying a change in the level of the gene product that mimics that observed in a dwarf mouse, a caloric-restricted mouse, or a dwarf mouse that is caloric-restricted relative to a control-fed normal mouse, thereby identifying an intervention that modulates a biomarker of longevity.

30. The method of claim 29, wherein the change in the level of the gene product is determined using an oligonucleotide-based high density array.

31. A method of identifying a biomarker of aging, the method comprising:
comparing an expression profile of a biological sample obtained from a dwarf mouse to the gene expression profile from a control-fed normal mouse and a caloric-restricted normal mouse, and identifying changes in the expression profile that occur in the dwarf mouse relative to the control-fed and caloric-restricted normal mice.

32. The method of claim 31, wherein the step of comparing the expression profile from the dwarf mouse to that of the control-fed and caloric restricted normal mice comprises measuring levels of RNA.

33. The method of claim 32, wherein the expression profile is determined using an oligonucleotide-based high density array.

34. The method of claim 31, wherein the step of comparing the expression profile from the dwarf mouse to that of the control-fed and caloric restricted normal mice comprises measuring levels of protein.

35. The method of claim 31, wherein the step of comparing the expression profile from the dwarf mouse to that of the control-fed and caloric restricted normal mice comprises measuring protein activity.

36. The method of claim 31, wherein the step of comparing the expression profile from the dwarf mouse to that of the control-fed and caloric restricted normal mice comprises measuring the levels of protein modification.

37. The method of claim 31, wherein the expression profile is obtained using liver tissue.

38. The method of claim 31, wherein the normal mouse is subjected to short-term caloric restriction.

39. A method of identifying an intervention that modulates a biomarker of longevity, the method comprising:
exposing a biological sample to a test intervention;
measuring the level of a gene product identified in accordance with claim 31; and
identifying a change in the level of the gene product that mimics that observed in a dwarf mouse, a caloric-restricted mouse, or a dwarf mouse that is caloric-restricted relative to a control-fed normal mouse, thereby identifying an intervention that modulates a biomarker of longevity.

40. The method of claim 39, wherein the change in the level of the gene product is determined using an oligonucleotide-based high density array.

41. A method of identifying a biomarker of aging, the method comprising:
comparing an expression profile from a caloric-restricted normal mouse to the gene expression profile from a control-fed dwarf mouse and a control-fed normal, and identifying changes in the expression profile that occur in the caloric-restricted mouse relative to the control-fed dwarf and normal mice.

42. The method of claim 41, wherein the step of comparing the expression profile from the caloric-restricted normal mouse to that of the control-fed dwarf and normal mice comprises measuring levels of RNA.

43. The method of claim 42, wherein the expression profile is determined using an oligonucleotide-based high density array.

44. The method of claim 41, wherein the step of comparing the expression profile from the caloric-restricted normal mouse to that of the control-fed dwarf and normal mice comprises measuring levels of protein.

45. The method of claim 41, wherein the step of comparing the expression profile from the caloric-restricted normal mouse to that of the control-fed dwarf and normal mice comprises measuring protein activity.

46. The method of claim 41, wherein the step of comparing the expression profile from the caloric-restricted normal mouse to that of the control-fed dwarf and normal mice comprises measuring the levels of protein modification.

47. The method of claim 41, wherein the expression profile is obtained using liver tissue.

48. The method of claim 41, wherein the normal mouse is subjected to short-term caloric restriction.

49. A method of identifying an intervention that modulates a biomarker of longevity, the method comprising:
exposing a biological sample to a test intervention;
measuring the level of a gene product identified in accordance with claim 41; and
identifying a change in the level of the gene product that mimics that observed in a dwarf mouse, a caloric-restricted mouse, or a dwarf mouse that is caloric-restricted relative to a control-fed normal mouse, thereby identifying an intervention that modulates a biomarker of longevity.

50. The method of claim 39, wherein the change in the level of the gene product is determined using an oligonucleotide-based high density array.


SEQUENCE LISTING (34 sequences)

SEQ ID NO: 1 - DNA, 24 bp, Artificial Sequence
  Description of Artificial Sequence CCAAT/enhancer binding protein, delta quantitative PCR Forward primer
cagttcttcaaaaaactgcccagc

SEQ ID NO: 2 - DNA, 22 bp, Artificial Sequence
  Description of Artificial Sequence CCAAT/enhancer binding protein, delta quantitative PCR Reverse primer
aaagaaactagcgattcgggcg

SEQ ID NO: 3 - DNA, 25 bp, Artificial Sequence
  Description of Artificial SequenceCell line NK14 derived transforming oncogene quantitative PCR Forward primer
tgattttctagcagcatacctggga

SEQ ID NO: 4 - DNA, 25 bp, Artificial Sequence
  Description of Artificial SequenceCell line NK14 derived transforming oncogene quantitative PCR Reverse primer
atcacaactgggtaaagacagcagg

SEQ ID NO: 5 - DNA, 23 bp, Artificial Sequence
  Description of Artificial SequenceCytochrome P450, 4a14 quantitative PCR Forward primer
ttgggccaaactgtgaaaaaatc

SEQ ID NO: 6 - DNA, 22 bp, Artificial Sequence
  Description of Artificial SequenceCytochrome P450, 4a14 quantitative PCR Reverse primer
attgccaaaactgctctggctc

SEQ ID NO: 7 - DNA, 23 bp, Artificial Sequence
  Description of Artificial SequenceCytochrome P450, 2f2 quantitative PCR Forward primer
gcttcctcacaaagatggcacag

SEQ ID NO: 8 - DNA, 21 bp, Artificial Sequence
  Description of Artificial SequenceCytochrome P450, 2f2 quantitative PCR Reverse primer
gtttctgtgccaccgaagagc

SEQ ID NO: 9 - DNA, 25 bp, Artificial Sequence
  Description of Artificial SequenceFatty acid synthase quantitative PCR Forward primer
ttgggttttgacttttctgcagctg

SEQ ID NO: 10 - DNA, 25 bp, Artificial Sequence
  Description of Artificial SequenceFatty acid synthase quantitative PCR Reverse primer
cacgtgcagtttaattgtgggatca

SEQ ID NO: 11 - DNA, 25 bp, Artificial Sequence
  Description of Artificial SequenceG0/G1 switch gene 2 quantitative PCR Forward primer
cagagctcagatggaaagtgtgcag

SEQ ID NO: 12 - DNA, 22 bp, Artificial Sequence
  Description of Artificial SequenceG0/G1 switch gene 2 quantitative PCR Reverse primer
tgcacaccgtctcaactaggcc

SEQ ID NO: 13 - DNA, 25 bp, Artificial Sequence
  Description of Artificial Sequence Phenylalanine glyoxalase 1 quantitative PCR Forward primer
ggtctgttaccttctggggtttcag

SEQ ID NO: 14 - DNA, 25 bp, Artificial Sequence
  Description of Artificial Sequence Phenylalanine glyoxalase 1 quantitative PCR Reverse primer
tgattccgaattgctctcaggagta

SEQ ID NO: 15 - DNA, 21 bp, Artificial Sequence
  Description of Artificial SequenceInsulin-like growth factor 1 (IGF1) quantitative PCR Forward primer
cacggagcagaaaatgccaca

SEQ ID NO: 16 - DNA, 20 bp, Artificial Sequence
  Description of Artificial SequenceInsulin-like growth factor 1 (IGF1) quantitative PCR Reverse primer
cattgggggaaatgcccatc

SEQ ID NO: 17 - DNA, 25 bp, Artificial Sequence
  Description of Artificial SequenceInsulin-like growth factor binding protein 2 quantitative PCR Forward primer
agtgctggtgtgtgaaccccaatac

SEQ ID NO: 18 - DNA, 25 bp, Artificial Sequence
  Description of Artificial SequenceInsulin-like growth factor binding protein 2 quantitative PCR Reverse primer
accagtctcctgctgctcgttgtag

SEQ ID NO: 19 - DNA, 22 bp, Artificial Sequence
  Description of Artificial SequenceLong-chain fatty-acyl elongase quantitative PCR Forward primer
catcgtccctggagctgaacag

SEQ ID NO: 20 - DNA, 25 bp, Artificial Sequence
  Description of Artificial SequenceLong-chain fatty-acyl elongase quantitative PCR Reverse primer
ccaggattatgtgtgaggtcgaaca

SEQ ID NO: 21 - DNA, 21 bp, Artificial Sequence
  Description of Artificial Sequence Metallothionein 1 quantitative PCR Forward primer
ctcctgcgcctgcaagaactg

SEQ ID NO: 22 - DNA, 21 bp, Artificial Sequence
  Description of Artificial Sequence Metallothionein 1 quantitative PCR Reverse primer
acacagccctgggcacatttg

SEQ ID NO: 23 - DNA, 21 bp, Artificial Sequence
  Description of Artificial Sequencep300/CBP-associated factor quantitative PCR Forward primer
gcttctgacatggaaggcatg

SEQ ID NO: 24 - DNA, 25 bp, Artificial Sequence
  Description of Artificial Sequencep300/CBP-associated factor quantitative PCR Reverse primer
accagtctgagacacttaatgcagc

SEQ ID NO: 25 - DNA, 24 bp, Artificial Sequence
  Description of Artificial SequencePeroxisome proliferator activated receptor alpha quantitative PCR Forward primer
cagtccccagtctggtcttaaccg

SEQ ID NO: 26 - DNA, 23 bp, Artificial Sequence
  Description of Artificial SequencePeroxisome proliferator activated receptor alpha quantitative PCR Reverse primer
ggaagggaacagaccgctcagac

SEQ ID NO: 27 - DNA, 25 bp, Artificial Sequence
  Description of Artificial SequenceQuiescin Q6 quantitative PCR Forward primer
tcagtgctctactcgtcctctgacc

SEQ ID NO: 28 - DNA, 22 bp, Artificial Sequence
  Description of Artificial SequenceQuiescin Q6 quantitative PCR Reverse primer
cacaccaggaggcgaagaactc

SEQ ID NO: 29 - DNA, 25 bp, Artificial Sequence
  Description of Artificial SequenceThyroid hormone responsive SPOT14 homolog (Rattus) quantitative PCR Forward primer
ccacctctgggatgtcgtttagtgc

SEQ ID NO: 30 - DNA, 22 bp, Artificial Sequence
  Description of Artificial SequenceThyroid hormone responsive SPOT14 homolog (Rattus) quantitative PCR Reverse primer
agggctttggattccgtgtttg

SEQ ID NO: 31 - DNA, 25 bp, Artificial Sequence
  Description of Artificial Sequence Transcription elongation factor A (SII) 1 quantitative PCR Forward primer
ccagctgaaatgtaggctgtagcaa

SEQ ID NO: 32 - DNA, 25 bp, Artificial Sequence
  Description of Artificial Sequence Transcription elongation factor A (SII) 1 quantitative PCR Reverse primer
acaggagtctgaacacaggcagaag

SEQ ID NO: 33 - DNA, 23 bp, Artificial Sequence
  Description of Artificial SequenceU2 small nuclear ribonucleoprotein auxiliary factor (U2AF), 65kDa quantitative PCR Forward primer
ttcccccatggtaggaacatagc

SEQ ID NO: 34 - DNA, 23 bp, Artificial Sequence
  Description of Artificial SequenceU2 small nuclear ribonucleoprotein auxiliary factor (U2AF), 65kDa quantitative PCR Reverse primer
agaacaggaaggaccagaagcca